(19) United States
(12) Patent Application Publication
(10) Pub. No.: US 2003/0105073 A1
(43) Pub. Date: Jun. 5, 2003

(54) Quinolone derivatives

(76) Inventors: Alan Findlay Haughan, Cambridge (GB); Hazel Joan Dyke, Saffron Walden (GB); George Martin Buckley, Biggleswade (GB); Natasha Davies, Cambridge (GB); Duncan Robert Hannah, Bury St. Edmonds (GB); Marianna Dilani Richard, Haverhill (GB); Andrew Sharpe, Cambridge (GB); Sophie Caroline Williams, Cambridge (GB)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 10/277,497

(22) Filed: Oct. 22, 2002

(30) Foreign Application Priority Data
Oct. 23, 2001 (GB) 0125365.7
Mar. 7, 2002 (GB) 0205372.6

(51) Int. Cl. A61K 31/551; A61K 31/541; A61K 31/5377; A61K 31/496; A61K 31/4709; C07D 417/02; C07D 413/02; C 07D 4 3/02

(52) U.S. Cl. 514/210.21; 514/218; 514/228.2; 514/233.5; 514/312; 514/253.07; 514/253.08; 544/60; 544/363; 546/156; 546/153; 540/575; 544/128

(57) ABSTRACT

Quinolone derivatives of formula (1) are described:

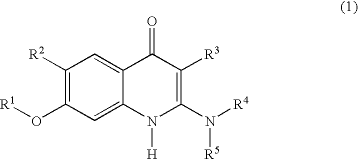

wherein:

X is an O or S atom;

R¹ is an aliphatic, cycloaliphatic, or cycloalkyl-alkyl-group;

R² is a —CN group or an optionally substituted heteroaromatic group;

R³ is a hydrogen atom or an alkyl, —CN, —CO₂H, —CO₂R⁶ or —CONR⁷R⁸ group;

R⁴ is a chain -Alk¹-L¹-Alk²-R⁹;

R⁵ is a hydrogen atom or an alkyl group;

or NR⁴R⁵ forms an optionally substituted heterocycloaliphatic ring optionally fused to an optionally substituted monocyclic C₆₋₁₂aromatic group or an optionally substituted monocyclic C₁₋₉heteroaromatic group; and the salts, solvates, hydrates, tautomers, isomers or N-oxides thereof.

The compounds are potent inhibitors of IMPDH and are of use as immunosuppressants, anti-cancer agents, anti-inflammatory agents, antipsoriatic and anti-viral agents.

This invention relates to a series of quinolones, to processes for their preparation, to pharmaceutical compositions containing them and to their use in medicine.

Inosine-5′-monophosphate dehydrogenase (IMPDH; EC 1.1.1.205) is an enzyme involved in the de novo synthesis of guanine nucleotides. IMPDH catalyses the β-nicotinamide adenine dinucleotide (NAD)-dependant oxidation of inosine-5′-monophosphate (IMP) to xanthosine-5′-monophosphate (XMP) (Jackson R. C. et al., Nature, 256, pp. 331-333, (1975)). Guanine nucleotides are essential to the cell for RNA and DNA synthesis, intermediates in signalling pathways and as energy sources for metabolic pathways.

IMPDH is ubiquitous in eukaryotes, bacteria and protozoa (Y. Natsumeda & S. F. Carr, Ann. N.Y. Acad., 696, pp. 88-93, (1993)). Two isoforms of human IMPDH, designated type I and type II, have been identified and sequenced (F. R. Collart and E. Huberman, J. Biol. Chem., 263, pp. 15769-15772, (1988); Y. Natsumeda et al J. Biol. Chem., 265, pp 5292-5295, (1990)). Each is 514 amino acids and they share 84% sequence identity. Both IMPDH type I and type II form active tetramers in solution, with subunit molecular weights of 56 kDa (Y. Yamada et. al., Biochemistry, 27, pp. 2737-2745, (1988)). It is thought that type I is the predominant isoform expressed in normal cells, whilst type II is upregulated in neoplastic and replicating cells. Studies have postulated that selective inhibition of type II IMPDH could provide a therapeutic advantage by reducing potential toxicity effects caused by inhibiting the type I isoform (Pankiewicz K. W, Expert Opin. Ther. Patents 11 (7) pp 1161-1170, (2001)).

The de novo synthesis of guanine nucleotides, and thus the activity of IMPDH, is particularly important in B and T-lymphocytes. These cells depend on the de novo, rather than the salvage pathway to generate sufficient levels of nucleotides necessary to initiate a proliferative response to mitogen or antigen (A. C. Allison et. al., Lancet II, 1179, (1975) and A. C. Allison et. al., Ciba Found. Symp., 48, 207, (1977)). Thus, IMPDH is an attractive target for selectively inhibiting the immune system without also inhibiting the proliferation of other cells.

Mycophenolic acid (MPA) and some of its derivatives have been described in U.S. Pat. Nos. 5,380,879 and 5,444,072 and PCT publications WO 94/01105 and WO 94/12184 as potent, uncompetitive, reversible inhibitors of human IMPDH type I (Kᵢ=33 nM) and type II (Kᵢ=9 nM). MPA has been demonstrated to block the response of B and T-cells to mitogen or antigen (A. C. Allison et. al., Ann. N. Y. Acad. Sci., 696, 63, (1993)).

Immunosuppressants, such as MPA, are useful drugs in the treatment of transplant rejection and autoimmune diseases. (R. E. Morris, Kidney Intl., 49, Suppl. 53, S-26, (1996)). However, MPA is characterized by undesirable pharmacological properties, such as gastrointestinal toxicity. (L. M. Shaw, et. al., Therapeutic Drug Monitoring, 17, pp. 690-699, (1995)).

Mycophenolate mofetil, a prodrug which quickly liberates free MPA in vivo, was recently approved to prevent acute allograft rejection following kidney transplantation (i.e. renal allograft failure) and heart transplantation. (L. M. Shaw, et. al., Therapeutic Drug Monitoring, 17, pp. 690-699, (1995); H. W. Sollinger, Transplantation, 60, pp. 225-232, (1995); J. Kobashigawa Transplant, 66, pp. 507, (1998)). Mycophenolate mofetil has also been used for the treatment of rheumatoid arthritis. The experimental use of mycophenolate mofetil in the treatment of systemic lupus erythematosus, lupus nephritis, myasthenia gravis, inflammatory eye disease, autoimmune and inflammatory skin disorders (including psoriasis) and glomerular disease has also been described (R. Bentley, Chem. Rev., 100, pp. 3801-3825, (2000)). Mycophenolate mofetil has also been postulated to be of use for the treatment of atopic dermatitis (Grundmann-Kollman M et al, Archives of Dermatology, 137 (7), pp. 870-873, (2001)) and has been shown to be effective in predictive animal models of multiple sclerosis (Tran G. T et al, International Immunopharmacology, 1 (9-10) pp. 1709-1723, (2001)).

Several clinical observations, however, limit the therapeutic potential of this drug. (L. M. Shaw, et. al., Therapeutic Drug Monitoring, 17, pp. 690-699, (1995)).

Nucleoside analogues such as tiazofurin, ribavirin and mizoribine also inhibit IMPDH (L. Hedstrom, et. al., Biochemistry, 29, pp. 849-854, (1990)). These nucleoside analogues are competitive inhibitors of IMPDH, but also inhibit other NAD dependant enzymes. This lack of specificity limits the therapeutic application of these compounds. New agents with improved selectivity for IMPDH would represent a significant improvement over these nucleoside analogues. Mizorbine (Bredinin®)) has been approved in Japan for multiple indications in transplantation and autoimmune diseases including prevention of rejection after renal transplantation, idiopathic glomerulonephritis, lupus nephritis and rheumatoid arthritis.

Vertex has recently disclosed a series of novel IMPDH inhibitors (WO 97/40028), of which VX-497 has been evaluated for the treatment of psoriasis.

It is also known that IMPDH plays a role in other metabolic events. Increased IMPDH activity has been observed in rapidly proliferating human leukemic cell lines and other tumour cell lines, indicating IMPDH as a target for anti-cancer as well as immunosuppressive chemotherapy (M. Nagai et. al., Cancer Res., 51, pp. 3886-3890, (1991), Pankiewicz K. W., Exp. Opin. Ther. Patents, 11, pp. 1161-1170, (2001)). IMPDH has also been shown to play a role in the proliferation of smooth muscle cells, indicating that inhibitors of IMPDH may be useful in preventing restenosis or other hyperproliferative vascular diseases (C. R. Gregory et. al., Transplantation, 59, pp. 655-61, (1995); PCT publication WO 94/12184; and PCT publication WO 94/01105).

Additionally, IMPDH has been shown to play a role in viral replication in some virus-infected cell lines. (S. F. Carr, J. Biol. Chem., 268, pp. 27286-27290, (1993)). VX-497 is currently being evaluated for the treatment of hepatitis C in humans.

Thus, there remains a need for potent IMPDH inhibitors with improved pharmacological properties. Such inhibitors would have therapeutic potential as immunosuppressants, anti-cancer agents, anti-inflammatory agents, antipsoriatic and anti-viral agents.

Japanese Patent Application number JP04164070 discloses the synthesis of a general class of quinolones for use as bactericides.

International Patent Application numbers WO-A-99/55677 and WO-A-00/21949 both disclose a general class of 2-aminoquinolones for use as inhibitors of methionyl t-RNA synthetase and antibacterial agents.

Co-pending International Patent Application number WO-A-01/81340 discloses a general class of heterocycles as inhibitors of IMPDH.

The present inventors disclose new potent IMPDH inhibitors based on substituted quinolone derivatives.

Thus according to one aspect of the invention we provide a compound of formula (1):

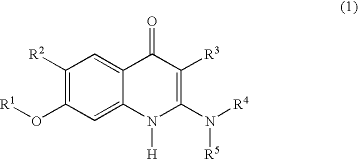

wherein:

X is an O or S atom;

R¹ is an aliphatic, cycloaliphatic or cycloalkyl-alkyl-group;

R² is a —CN group or an optionally substituted heteroaromatic group;

R³ is a hydrogen atom or an alkyl, —CN, —CO₂H, —CO₂R⁶ or —CONR⁷R⁸ group, in which R⁶ is an alkyl group and R⁷ and R⁸, which may be the same or different, is each a hydrogen atom or an alkyl group;

R⁴ is a chain -Alk¹-L¹-Alk²-R⁹ in which Alk¹ is a covalent bond or an optionally substituted aliphatic chain, L¹ is a covalent bond or a linker atom or group, Alk² is a covalent bond or a C₁₋₃ alkylene chain and R⁹ is a hydrogen atom or an optionally substituted cycloaliphatic, heterocycloaliphatic, aromatic or heteroaromatic group; provided that R⁴ is not a hydrogen atom;

R⁵ is a hydrogen atom or an alkyl group;

or NR⁴R⁵ forms an optionally substituted heterocycloaliphatic ring optionally fused to an optionally substituted monocyclic C₆₋₁₂aromatic group or an optionally substituted monocyclic C₁₋₉heteroaromatic group;

and the salts, solvates, hydrates, tautomers, isomers or N-oxides thereof;

provided that the compound of formula (1) is other than:

7-methoxy-2-methylamino-6-oxazol-5-yl-1H-quinolin-4-one or

2-dimethylamino-7-methoxy-6-oxazol-5-yl-1H-quinolin-4-one.

It will be appreciated that certain compounds of formula (1) may exist as geometric isomers (E or Z isomers). The compounds may also have one or more chiral centres, and exist as enantiomers or diastereomers. The invention is to be understood to extend to all such geometric isomers, enantiomers, diastereomers and mixtures thereof, including racemates. Formula (1) and the formulae hereinafter are intended to represent all individual isomers and mixtures thereof, unless stated or shown otherwise. In addition, compounds of formula (1) may exist as tautomers, for example keto (CH₂C═O)— enol (CH═CHOH) tautomers. Quinolones may also exist as tautomers; one possible example is illustrated below:

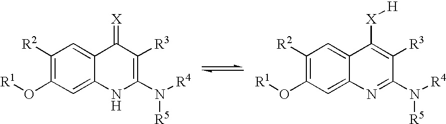

Formula (1) and the formulae hereinafter are intended to represent all individual tautomers and mixtures thereof, unless stated otherwise.

It will also be appreciated that where desired the compounds of the invention may be administered in a pharmaceutically acceptable pro-drug form, for example, as a protected carboxylic acid derivative, e.g. as an acceptable ester. It will be further appreciated that the pro-drugs may be converted in vivo to the active compounds of formula (1), and the invention is intended to extend to such pro-drugs. Such prodrugs are well known in the literature, see for example International Patent Application No. WO 00/23419, Bodor N. (Alfred Benson Symposium, 1982, 17, 156-177), Singh G. et al (J. Sci. Ind. Res., 1996, 55, 497-510) and Bundgaard H. (Design of Prodrugs, 1985, Elsevier, Amsterdam).

In the compounds of the invention as represented by formula (1) and the more detailed description hereinafter certain of the general terms used in relation to substituents are to be understood to include the following atoms or groups unless specified otherwise.

The term “aliphatic group” is intended to include optionally substituted straight or branched C₁₋₁₀alkyl, e.g. C₁₋₆ alkyl, C₂₋₁₀alkenyl e.g. C₂₋₆alkenyl or C₂₋₁₀alkynyl e.g. C₂₋₆alkynyl groups. Optional substituents when present on these groups include those optional substituents mentioned hereinafter.

Thus as used herein the term “alkyl”, whether present as a group or part of a group includes straight or branched C₁₋₁₀alkyl groups, for example C₁₋₆alkyl groups such as methyl, ethyl, n-propyl, i-propyl, n-butyl, i-butyl, s-butyl or t-butyl groups. Optional substituents when present on these groups include those optional substituents mentioned hereinafter.

The terms “alkenyl” or “alkynyl” are intended to mean straight or branched C₂₋₁₀alkenyl or C₂₋₁₀alkynyl groups such as C₂₋₆alkenyl or C₂₋₆alkynyl groups such as —CHCH₂, —CHCHCH₃, —CH₂CHCHCH₃, —CCH, —CH₂CCH and —CH₂CCCH₃ groups. Such groups may be substituted by those optional substituents mentioned hereinafter.

Particular examples of aliphatic groups include optionally substituted C₁₋₆ alkyl groups such as —CH₃, —CH₂CH₃, —CH(CH₃)₂, —(CH₂)₂CH₃, —(CH₂)₃CH₃, —CH(CH₃)CH₂CH₃, —CH₂CH(CH₃)₂, —CH₂C(CH₃)₃, —C(CH₃)₃, —(CH₂)₄CH₃, —(CH₂)₅CH₃, or C₂₋₆alkenyl or C₂₋₆alkynyl groups such as —CHCH₂, —CHCHCH₃, —CH₂CHCH₂, —CHCHCH₂CH₃, —CH₂CHCHCH₃, —(CH₂)₂CHCH₂, —CCH, —CCCH₃, —CH₂CCH, —CCCH₂CH₃, —CH₂CCCH₃, or —(CH₂)₂CCH groups.

The term “aliphatic chain” is intended to include those alkyl, alkenyl or alkynyl groups as just described where a terminal hydrogen atom is replaced by a covalent bond to give a divalent chain.

Examples of aliphatic chains include optionally substituted C₁₋₆ alkylene chains such as —CH₂—, —CH₂CH₂—, —CH(CH₃)CH₂—, —(CH₂)₂CH₂—, —(CH₂)₃CH₂—, —CH(CH₃)(CH₂)₂CH₂—, —CH₂CH(CH₃)CH₂—, —C(CH₃)₂—, —C(CH₃)₂CH₂—, —CH₂C(CH₃)₂CH₂—, —(CH₂)₂CH(CH₃)CH₂—, —CH(CH₃)CH₂CH₂—, —CH(CH₃)CH₂CH(CH₃)CH₂—, —CH₂CH(CH₃)CH₂CH₂—, —(CH₂)₂C(CH₃)₂—, —(CH₂)₄CH₂—, —(CH₂)₅CH₂, or C₂₋₆alkenylene or C₂₋₆alkynylene chains such as —CHCH—, —CHCHCH₂ —CH₂CHCH—, —CHCHCH₂CH₂—, —CH₂CHCHCH₂—, —(CH₂)₂CHCH—, —CC—, —CCCH₂, —CH₂CC—, —CCCH₂CH₂—, —CH₂CCCH₂— or —(CH₂)₂CCH— chains. More particular examples include optionally substituted C₁₋₃ alkylene chains selected from —CH₂—, —CH₂CH₂—, —CH₂CH₂CH₂—, —CH(CH₃)CH₂—, —C(CH₃)₂— and —CH₂CH(CH₃)— chains.

The term “cycloaliphatic group” includes optionally substituted non-aromatic cyclic or multicyclic, saturated or partially saturated C₃₋₁₀ ring systems, such as, cyclopropyl, cyclobutyl, cyclopentyl, cyclohexyl, cycloheptyl, cyclooctyl, cyclobutenyl, cyclopentenyl, cyclohexenyl, cycloheptenyl, cyclooctenyl, adamantyl, norbornyl, norbornenyl, bicyclo[2.2.1]heptanyl or bicyclo[2.2.1]heptenyl. Particular examples include optionally substituted C₃₋₆ cycloalkyl ring systems such as cyclopropyl, cyclobutyl, cyclopentyl and cyclohexyl groups. Optional substituents present on those groups include those substituents mentioned hereinafter.

The term “cycloalkyl-alkyl-group” refers to a C₁₋₆ alkyl group (as described herein) where a terminal hydrogen atom is replaced by a C₃₋₆ cycloalkyl ring (as described herein). Examples include —(CH₂)₁₋₆-cyclopropyl, —(CH₂)₁₋₆-cyclobutyl, —(CH₂)₁₋₆-cyclopentyl or —(CH₂)₁₋₆-cyclohexyl.

The term “heterocycloaliphatic group” refers to an optionally substituted 3 to 10 membered saturated or partially saturated monocyclic or saturated or partially saturated multicyclic hydrocarbon ring system containing one, two, three or four L² linker atoms or groups. Particular examples of suitable L² atoms or groups include —O— or —S— atoms or —C(O)—, —C(O)O—, —OC(O)—, —C(S)—, —S(O)—, —S(O)₂—, —N(R¹⁰)— [where R¹⁰ is a hydrogen atom or a C₁₋₆ alkyl group], —N(R¹⁰)N(R¹⁰)—, —N(R¹⁰)O—, —ON(R¹⁰)—, —CON(R¹⁰)—, —OC(O)N(R¹⁰)—, —CSN(R¹⁰)—, —N(R¹⁰)CO—, —N(R¹⁰)C(O)O—, —N(R¹⁰)CS—, —S(O)₂N(R¹⁰)—, —N(R¹⁰)S(O)₂—, —N(R¹⁰)CON(R¹⁰)—, —N(R¹⁰)CSN(R¹⁰)—, or —N(R¹⁰)SO₂N(R¹⁰)— groups. Where the linker group contains two R¹⁰ substituents, these may be the same or different. Optional substituents present on the heterocycloaliphatic groups include those substituents mentioned hereinafter.

Particular examples of heterocycloaliphatic groups include optionally substituted cyclobutanonyl, cyclopentanonyl, cyclohexanonyl, azetidinyl, tetrahydrofuranyl, tetrahydropyranyl, pyrrolinyl, e.g. 2- or 3-pyrrolinyl, pyrrolidinyl, pyrrolidinonyl, oxazolidinyl, oxazolidinonyl, dioxolanyl, e.g. 1,3-dioxolanyl, imidazolinyl, e.g. 2-imidazolinyl, imidazolidinyl, pyrazolinyl, e.g. 2-pyrazolinyl, pyrazolidinyl, thiazolinyl, thiazolidinyl, pyranyl, e.g. 2- or 4-pyranyl, pyranonyl, piperidinyl, piperidinonyl, quinuclidinyl, 1,4-dioxanyl, morpholinyl, morpholinonyl, 1,4-dithianyl, thiomorpholinyl, piperazinyl, N—C₁₋₆ alkylpiperazinyl, homopiperazinyl, dihydrofuran-2-onyl, tetrahydropyran-2-onyl, isothiazolidinyl 1,1-dioxide, [1,2]thiazinanyl 1,1-dioxide, tetrahydrothiophenyl, tetrahydrothiopyranyl, pyrazolidin-3-onyl, tetrahydrothiopyranyl 1,1-dioxide, tetrahydrothiophenyl 1,1-dioxide, 1,3,5-trithianyl, oxazinyl, e.g. 2H-1,3-, 6H-1,3-, 6H-1,2-, 2H-1,2- or 4H-1,4-oxazinyl, 1,2,5-oxathiazinyl, isoxazinyl, e.g. o- or p-isoxazinyl, oxathiazinyl, e.g. 1,2,5 or 1,2,6-oxathiazinyl, or 1,3,5,-oxadiazinyl groups.

Cycloaliphatic groups may be linked to the remainder of the compound of formula (1) by any available ring carbon atom. Heterocycloaliphatic groups may be linked to the remainder of the compound of formula (1) by any available ring carbon or, where available, ring nitrogen atom.

For the case where NR⁴R⁵ represents an optionally substituted heterocycloaliphatic ring, the heterocyclic moiety must contain at least one nitrogen atom. This includes, for example, azetidinyl, pyrrolidinyl, piperidinyl, imidazolidinyl, thiazolidinyl, pyrazolidinyl, piperazinyl, N—C₁₋₆ alkylpiperazinyl, homopiperazinyl, morpholinyl, thiomorpholinyl, oxazolidinyl and the like.

The NR⁴R⁵ heterocycloaliphatic ring may optionally be fused to an optionally substituted monocyclic C₆₋₁₂aromatic group, such as phenyl or an optionally substituted monocyclic C₁₋₉heteroaromatic group containing for example one, two, three or four heteroatoms selected from oxygen, sulfur or nitrogen atoms.

The optional substituents which may be present on the aliphatic, alkyl, alkenyl, alkynyl, cycloaliphatic or heterocycloaliphatic groups, described above and generally herein include one, two, three or more substituents, which each may be the same or different, selected from halogen atoms, or alkoxy, haloalkyl, haloalkoxy, hydroxy (—OH), thiol (—SH), alkylthio, amino (—NH₂), substituted amino, optionally substituted C₆₋₁₂arylamino, —CN, —CO₂H, —CO₂R¹¹ (where R¹¹ is an optionally substituted C₁₋₆ alkyl group), —SO₃H, —SOR¹² (where R¹² is a C₁₋₆ alkyl group) —SO₂R¹², —SO₃R¹², —OCO₂R¹², —C(O)H, —C(O)R¹², —OC(O)R¹², —C(S)R¹², —C(O)N(R¹³)(R¹⁴) (where R¹³ and R¹⁴, which may be the same or different is each a hydrogen atom or a C₁₋₆ alkyl group), —OC(O)N(R¹³)(R¹⁴), —N(R¹³)C(O)R¹⁴, —CSN(R¹³)(R¹⁴), —N(R¹³)C(S)(R¹⁴), —SO₂N(R¹³)(R¹⁴), —N(R¹³)SO₂R¹⁴, —N(R¹³)C(O)N(R¹⁴)(R¹⁵) (where R¹⁵ is a hydrogen atom or a C₁₋₆ alkyl group), —N(R¹³)C(S)N(R¹⁴)(R¹⁵), —N(R¹³)SO₂N(R¹⁴)(R¹⁵), or an optionally substituted aromatic or heteroaromatic group or a C₁₋₆ alkyl group optionally substituted by one, two, three or more of the same or different halogen atoms, or alkoxy, haloalkyl, haloalkoxy, hydroxy (—OH), thiol (—SH), alkylthio, amino (—NH₂), substituted amino, optionally substituted C₆₋₁₂arylamino, —CN, —CO₂H, —CO₂R¹¹, —SO₃H, —SOR¹², —SO₂R¹², —SO₃R¹², —OCO₂R¹², —C(O)H, —C(O)R¹², —OC(O)R¹², —C(S)R¹², —C(O)N(R¹³)(R¹⁴), —OC(O)N(R¹³)(R¹⁴), —N(R¹³)C(O)R¹⁴, —CSN(R¹³)(R¹⁴), —N(R¹³)C(S)(R¹⁴), —SO₂N(R¹³)(R¹⁴), —N(R¹³)SO₂R¹⁴, —N(R¹³)C(O)N(R¹⁴)(R¹⁵), —N(R¹³)C(S)N(R¹⁴)(R¹⁵), —N(R¹³)SO₂N(R¹⁴)(R¹⁵) or optionally substituted aromatic or heteroaromatic groups. Substituted amino groups include —NHR¹² and —N(R¹²)(R¹³) groups.

The optional substituents which may be present on aliphatic chains represented by Alk¹ or Alk² include one, two, three or more substituents where each substituent may be the same or different and is selected from halogen atoms, e.g. fluorine, chlorine, bromine or iodine atoms, or —OH, —CO₂H, —CO₂R¹⁶ [where R¹⁶ is an optionally substituted straight or branched C₁₋₆ alkyl group], e.g. —CO₂CH₃ or —CO₂C(CH₃)₃, —CONHR¹⁶, e.g. —CONHCH₃, —CON(R¹⁶)₂, e.g. —CON(CH₃)₂, —COR¹⁶, e.g. —COCH₃, C₁₋₆alkoxy, e.g. methoxy or ethoxy, haloC₁₋₆alkoxy, e.g. trifluoromethoxy or difluoromethoxy, thiol (—SH), —S(O)R¹⁶, e.g. —S(O)CH₃, —S(O)₂R¹⁶, e.g. —S(O))₂CH₃, C₁₋₆alkylthio e.g. methylthio or ethylthio, amino, —NHR¹⁶, e.g. —NHCH₃ or —N(R¹⁶)₂, e.g. —N(CH₃)₂ groups. Where two R¹⁶ groups are present in any of the above substituents these may be the same or different.

When R¹⁰, R¹², R¹³, R¹⁴, R¹⁵ or R¹⁶ is present as a C₁₋₆alkyl group it may be a straight or branched C₁₋₆ alkyl group e.g. a C₁₋₃ alkyl group such as methyl, ethyl or i-propyl. Optional substituents which may be present on R¹⁶ include for example one, two or three substituents which may be the same or different selected from fluorine, chlorine, bromine or iodine atoms or hydroxy or C₁₋₆ alkoxy e.g. methoxy or ethoxy groups.

When L¹ is present in compounds of formula (1) as a linker atom or group it may be any such atom or group as hereinbefore described in relation to L² linker atoms and groups. When in compounds of this type Alk¹ is a covalent bond then L¹ is a —C(O)—, —C(O)O—, —C(S)—, —S(O))₂—, —CON(R¹⁰)—, —CSN(R¹⁰)— or —S(O))₂N(R¹⁰)— group, where R¹⁰ is as herein defined.

The term “halogen atom” is intended to include fluorine, chlorine, bromine or iodine atoms.

The term “haloalkyl” is intended to include the alkyl groups just mentioned substituted by one, two or three of the halogen atoms just described. Particular examples of such groups include —CF₃, —CCl₃, —CHF₂, —CHCl₂, —CH₂F, and —CH₂Cl groups.

The term “alkoxy” as used herein is intended to include straight or branched C₁₋₁₀alkoxy for example C₁₋₆alkoxy such as methoxy, ethoxy, n-propoxy, i-propoxy and t-butoxy. “Haloalkoxy” as used herein includes any of those alkoxy groups substituted by one, two or three halogen atoms as described above. Particular examples include —OCF₃, —OCCl₃, —OCHF₂, —OCHCl₂, —OCH₂F and —OCH₂Cl groups.

As used herein the term “alkylthio” is intended to include straight or branched C₁₋₁₀alkylthio, e.g. C₁₋₆alkylthio such as methylthio or ethylthio groups.

The terms “aromatic group” and “aryl group” are intended to include for example optionally substituted monocyclic ring C₆₋₁₂ aromatic groups, such as phenyl, or bicyclic fused ring C₆₋₁₂ aromatic groups, such as, 1- or 2-naphthyl groups.

The terms “heteroaromatic group” and “heteroaryl group” are intended to include for example optionally substituted C₁₋₉ heteroaromatic groups containing for example one, two, three or four heteroatoms selected from oxygen, sulfur or nitrogen atoms. In general, the heteroaromatic groups may be for example monocyclic or bicyclic fused ring heteroaromatic groups. Monocyclic heteroaromatic groups include for example five- or six-membered heteroaromatic groups containing one, two, three or four heteroatoms selected from oxygen, sulfur or nitrogen atoms. Bicyclic heteroaromatic groups include for example eight- to thirteen-membered fused-ring heteroaromatic groups containing one, two or more heteroatoms selected from oxygen, sulfur or nitrogen atoms.

Each of these aromatic or heteroaromatic groups may be optionally substituted by one, two, three or more R¹⁷ atoms or groups as defined below.

Particular examples of monocyclic ring heteroaromatic groups of this type include pyrrolyl, furyl, thienyl, imidazolyl, N—C₁₋₆alkylimidazolyl, oxazolyl, isoxazolyl, thiazolyl, isothiazolyl, pyrazolyl, triazolyl, oxadiazolyl, thiadiazolyl, pyridyl, pyrimidinyl, pyridazinyl, pyrazinyl, tetrazolyl, or triazinyl.

Particular examples of bicyclic ring heteroaromatic groups of this type include benzofuryl, benzothienyl, benzotriazolyl, indolyl, indazolinyl, benzimidazolyl, imidazo[1,2-a]pyridyl, benzothiazolyl, benzoxazolyl, benzisoxazolyl, benzopyranyl, quinazolinyl, quinoxalinyl, naphthyridinyl, pyrido[3,4-b]pyridyl, pyrido[3,2-b]pyridyl, pyrido[4,3-b]-pyridyl, quinolinyl, isoquinolinyl or phthalazinyl.

The R² or R⁹ heteroaromatic groups may be attached to the remainder of the compound of formula (1) by any carbon or hetero e.g. nitrogen atom as appropriate.

Optional substituents which may be present on the aromatic or heteroaromatic groups include one, two, three or more substituents, each selected from an atom or group R¹⁷ in which R¹⁷ is —R^{17a} or -Alk³(R^{17a})_{f}, where R^{17a} is a halogen atom, or an amino (—NH₂), substituted amino, nitro, cyano, hydroxyl (—OH), substituted hydroxyl, amidino, formyl, carboxyl (—CO₂H), esterified carboxyl, thiol (—SH), substituted thiol, —COR¹⁸ [where R¹⁸ is an -Alk³(R^{17a})_{f}, heterocycloaliphatic, cycloaliphatic, aryl or heteroaryl group], —CSR¹⁸, —SO₃H, —SOR¹⁸, —SO₂R¹⁸, —SO₃R¹⁸, —SO₂NH₂, —SO₂NHR¹⁸, SO₂N(R¹⁸)₂, —CON₂, —CSNH₂, —CONHR¹⁸, —CSNHR¹⁸, —CON(R¹⁸)₂, —CSN(R¹⁸)₂, —N(R¹⁹)SO₂R¹⁸, [where R¹⁹ is a hydrogen atom or an alkyl group] —N(SO₂R¹⁸)₂, —N(R¹⁹)SO₂NH₂, —N(R¹⁹)SO₂NHR¹⁸, —N(R¹⁸)SO₂N(R¹⁹)₂, —N(R¹⁹)COR¹⁸, —N(R¹⁹)CONH₂, —N(R¹⁹)CONHR¹⁸, —N(R¹⁹)CON(R¹⁸)₂, —N(R¹⁹)CSNH₂, —N(R¹⁹)CSNHR¹⁸, —N(R¹⁹)CSN(R¹⁸)₂, —N(R¹⁹)CSR¹⁸, —N(R¹⁹)C(O)OR¹⁸, —SO₂NHet¹ [where —NHet¹ is an optionally substituted C₅₋₇cyclicamino group optionally containing one or more other —O— or —S— atoms or —N(R¹⁹)—, —C(O)— or —C(S)— groups], —CONHet¹, —CSNHet¹, —N(R¹⁹)SO₂NHet¹, —N(R¹⁹)CONHet¹, —N(R¹⁹)CSNHet¹, —SO₂N(R¹⁹)Het² [where Het² is an optionally substituted monocyclic C₅₋₇carbocyclic group optionally containing one or more —O— or —S— atoms or —N(R¹⁹)—, —C(O)— or —C(S)— groups], -Het², —CON(R¹⁹)Het², —CSN(R¹⁹)Het², —N(R¹⁹)CON(R¹⁹)Het², —N(R¹⁹)CSN(R¹⁹)Het², aryl or heteroaryl group; Alk³ is a straight or branched C₁₋₆alkylene, C₂₋₆alkenylene or C₂₋₆alkynylene chain, optionally interrupted by one, two or three —O— or —S— atoms or —S(O)_{g}— [where g is an integer 1 or 2] or —N(R¹⁹)— groups; and f is zero or an integer 1, 2 or 3. It will be appreciated that when two R¹⁸ or R¹⁹ groups are present in one of the above substituents, the R¹⁸ or R¹⁹ groups may be the same or different.

When in the group -Alk³(R^{17a})_{f} f is an integer 1, 2 or 3, it is to be understood that the substituent or substituents R^{17a} may be present on any suitable carbon atom in -Alk³. Where more than one R^{17a} substituent is present these may be the same or different and may be present on the same or different atom in -Alk³. Clearly, when f is zero and no substituent R^{17a} is present the chain represented by Alk³ becomes a corresponding group.

When R^{17a} is a substituted amino group it may be for example a group —NHR¹⁸ [where R¹⁸ is as defined above] or a group —N(R¹⁸)₂ wherein each R¹⁸ group is the same or different.

When R^{17a} is a substituted hydroxyl or substituted thiol group it may be for example a group —OR¹⁸ or a —SR¹⁸ group respectively.

Esterified carboxyl groups represented by the group R^{17a} include groups of formula —CO₂Alk⁴ wherein Alk⁴ is an optionally substituted alkyl group.

When Alk³ is present in or as a substituent it may be for example a methylene, ethylene, n-propylene, i-propylene, n-butylene, i-butylene, s-butylene, t-butylene, ethenylene, 2-propenylene, 2-butenylene, 3-butenylene, ethynylene, 2-propynylene, 2-butynylene or 3-butynylene chain, optionally interrupted by one, two, or three —O— or —S—, atoms or —S(O)—, —S(O))₂— or —N(R¹⁹)— groups.

When —NHet¹ or -Het² forms part of a substituent R¹⁷ each may be for example an optionally substituted 2- or 3-pyrrolinyl, pyrrolidinyl, pyrazolinyl, pyrazolidinyl, piperazinyl, imidazolinyl, imidazolidinyl, morpholinyl, thiomorpholinyl, piperidinyl, oxazolidinyl or thiazolidinyl group. Additionally Het² may represent for example, an optionally substituted cyclopentyl or cyclohexyl group. Optional substituents which may be present on —NHet¹ or -Het² include those substituents described above in relation to aromatic groups.

Particularly useful atoms or groups represented by R¹⁷ include fluorine, chlorine, bromine or iodine atoms, or C₁₋₆alkyl, e.g. methyl, ethyl, n-propyl, i-propyl, n-butyl or t-butyl, optionally substituted phenyl, pyridyl, pyrimidinyl, pyrrolyl, furyl, thiazolyl, thienyl, morpholinyl, thiomorpholinyl, piperazinyl, pyrrolidinyl or piperidinyl, C₁₋₆hydroxyalkyl, e.g. hydroxymethyl or hydroxyethyl, carboxyC₁₋₆alkyl, e.g. carboxyethyl, C₁₋₆alkylthio e.g. methylthio or ethylthio, carboxyC₁₋₆alkylthio, e.g. carboxymethylthio, 2-carboxyethylthio or 3-carboxypropylthio, C₁₋₆alkoxy, e.g. methoxy or ethoxy, hydroxyC₁₋₆alkoxy, e.g. 2-hydroxyethoxy, optionally substituted phenoxy, pyridyloxy, thiazolyoxy, phenylthio or pyridylthio, C₅₋₇cycloalkoxy, e.g. cyclopentyloxy, haloC₁₋₆alkyl, e.g. trifluoromethyl, haloC₁₋₆alkoxy, e.g. trifluoromethoxy, C₁₋₆alkylamino, e.g. methylamino or ethylamino, amino (—NH₂), aminoC₁₋₆alkyl, e.g. aminomethyl or aminoethyl, C₁₋₆dialkylamino, e.g. dimethylamino or diethylamino, aminoC₁₋₆alkylamino e.g. aminoethylamino, Het¹NC₁₋₆alkylamino e.g. morpholinopropylamino, C₁₋₆alkylaminoC₁₋₆alkyl, e.g. ethylaminoethyl, C₁₋₆dialkylaminoC₁₋₆alkyl, e.g. diethylaminoethyl, aminoC₁₋₆alkoxy, e.g. aminoethoxy, C₁₋₆alkylaminoC₁₋₆alkoxy, e.g. methylaminoethoxy, C₁₋₆dialkylaminoC₁₋₆alkoxy, e.g. dimethylaminoethoxy, diethylaminoethoxy, diisopropylaminoethoxy, or dimethylaminopropoxy, hydroxyC₁₋₆alkylamino e.g. hydroxyethylamino, imido, such as phthalimido or naphthalimido, e.g. 1,8-naphthalimido, nitro, cyano, amidino, formyl [HC(O)—], carboxyl (—CO₂H), —CO₂Alk⁴ [where Alk⁴ is as defined above], C₁₋₆alkanoyl e.g. acetyl, optionally substituted benzoyl, thiol (—SH), thioC₁₋₆alkyl, e.g. thiomethyl or thioethyl, —SC(═NH)NH₂, sulphonyl (—SO₃H), —SO₃R¹⁸, C₁₋₆alkylsulphinyl e.g. methylsulphinyl, C₁₋₆alkylsulphonyl, e.g. methylsulphonyl, aminosulphonyl (—SO₂NH₂), C₁₋₆alkylaminosulphonyl, e.g. methylamino-sulphonyl or ethylaminosulphonyl, C₁₋₆dialkylaminosulphonyl, e.g. dimethyl-aminosulphonyl or diethylaminosulphonyl, optionally substituted phenylamino-sulphonyl, carboxamido (—CONH₂), C₁₋₆alkylaminocarbonyl, e.g. methylamino-carbonyl or ethylaminocarbonyl, C₁₋₆dialkylaminocarbonyl, e.g. dimethyl-aminocarbonyl or diethylaminocarbonyl, aminoC₁₋₆alkylaminocarbonyl, e.g. aminoethylaminocarbonyl, C₁₋₆dialkylaminoC₁₋₆alkylaminocarbonyl, e.g. diethylaminoethylaminocarbonyl, aminocarbonylamino, C₁₋₆alkylaminocarbonylamino, e.g. methylaminocarbonylamino or ethylaminocarbonylamino, C₁₋₆dialkylaminocarbonylamino, e.g. dimethylaminocarbonylamino or diethylaminocarbonylamino, C₁₋₆alkylaminocabonylC₁₋₆alkylamino, e.g. methylaminocarbonylmethylamino, aminothiocarbonylamino, C₁₋₆alkylaminothiocarbonylamino, e.g. methylaminothiocarbonylamino or ethylaminothiocarbonylamino, C₁₋₆dialkylaminothiocarbonylamino, e.g. dimethylaminothiocarbonylamino or diethylaminothiocarbonylamino, C₁₋₆alkylaminothiocarbonylC₁₋₆alkylamino, e.g. ethylaminothiocarbonylmethylamino, —CONHC(═NH)NH₂, C₁₋₆alkylsulphonylamino, e.g. methylsulphonylamino or ethylsulphonylamino, C₁₋₆dialkylsulphonylamino, e.g. dimethylsulphonylamino or diethylsulphonylamino, optionally substituted phenylsulphonylamino, aminosulphonylamino (—NHSO₂NH₂), C₁₋₆alkylaminosulphonylamino, e.g. methylaminosulphonylamino or ethylaminosulphonylamino, C₁₋₆dialkylaminosulphonylamino, e.g. dimethylaminosulphonylamino or diethylaminosulphonylamino, optionally substituted morpholinesulphonylamino or morpholinesulphonylC₁₋₆alkylamino, optionally substituted phenylaminosulphonylamino, C₁₋₆alkanoylamino, e.g. acetylamino, aminoC₁₋₆alkanoylamino e.g. aminoacetylamino, C₁₋₆dialkylaminoC₁₋₆alkanoylamino, e.g. dimethylaminoacetylamino, C₁₋₆alkanoylaminoC₁₋₆alkyl, e.g. acetylaminomethyl, C₁₋₆alkanoylaminoC₁₋₆alkylamino, e.g. acetamidoethylamino, C₁₋₆alkoxycarbonylamino, e.g. methoxycarbonylamino, ethoxycarbonylamino or t-butoxycarbonylamino or optionally substituted benzyloxy, benzylamino, pyridylmethoxy, thiazolylmethoxy, benzyloxycarbonylamino, benzyloxycarbonylaminoC₁₋₆alkyl e.g. benzyloxycarbonylaminoethyl, thiobenzyl, pyridylmethylthio or thiazolylmethylthio groups.

Where desired, two R¹⁷ substituents may be linked together to form a cyclic group such as a cyclic ether, e.g. a C₁₋₆alkylenedioxy group such as methylenedioxy or ethylenedioxy.

It will be appreciated that where two or more R¹⁷ substituents are present, these need not necessarily be the same atoms and/or groups. In general, the substituent(s) may be present at any available ring position in the aromatic or heteroaromatic group.

The presence of certain substituents in the compounds of formula (1) may enable salts of the compounds to be formed. Suitable salts include pharmaceutically acceptable salts, for example acid addition salts derived from inorganic or organic acids, and salts derived from inorganic and organic bases.

Acid addition salts include hydrochlorides, hydrobromides, hydroiodides, alkylsulphonates, e.g. methanesulphonates, ethanesulphonates, or isothionates, arylsulphonates, e.g. p-toluenesulphonates, besylates or napsylates, phosphates, sulphates, hydrogen sulphates, acetates, trifluoroacetates, propionates, citrates, maleates, fumarates, malonates, succinates, lactates, oxalates, tartrates and benzoates.

Salts derived from inorganic or organic bases include alkali metal salts such as sodium or potassium salts, alkaline earth metal salts such as magnesium or calcium salts, and organic amine salts such as morpholine, piperidine, dimethylamine or diethylamine salts.

Particularly useful salts of compounds according to the invention include pharmaceutically acceptable salts, especially acid addition pharmaceutically acceptable salts.

Examples of optionally substituted alkyl groups present in ester groups of formulae —CO₂R¹¹ and —CO₂Alk⁴ include C₁₋₆ alkyl groups as herein described, in particular C₁₋₃ alkyl groups. Optional substituents, which may be present on these alkyl groups, include optionally substituted cycloaliphatic, aromatic or heteroaromatic groups as herein defined. Particular examples include optionally substituted C₃₋₆ cycloalkyl wherein the optional substituents include for example one, two or three substituents which may be the same or different selected from fluorine, chlorine, bromine or iodine atoms or hydroxy or C₁₋₆ alkoxy e.g. methoxy or ethoxy groups; or optionally substituted phenyl or five or six membered heteroaryl groups wherein the optional substituents include for example one, two or three substituents which may be the same or different selected from fluorine, chlorine, bromine, straight or branched C₁₋₆ alkyl, methoxy, OCF₃, OCF₂H, CF₃, CN, NHCH₃, N(CH₃)₂, CONH₂, CONHCH₃, CON(CH₃)₂, CO₂CH₃, CO₂CH₂CH₃, —CO₂C(CH₃)₃, or —COCH₃, —NHCOCH₃, —N(CH₃)COCH₃ or CO₂H.

Examples of alkyl groups, represented by R³, R⁵, R⁶, R⁷ or R⁸ include C₁₋₆ alkyl groups as herein described. More particular examples include C₁₋₃ alkyl groups, such as —CH₃, —CH₂CH₃, —CH₂CH₂CH₃ or —CH(CH₃)CH₃.

One particular group of compounds of the invention has the formula (1) wherein X is an O atom.

A particular group of compounds has the formula (1) wherein R³ is a hydrogen atom or a —CN group, especially a hydrogen atom.

A particularly useful group of compounds of the invention has the formula (2):

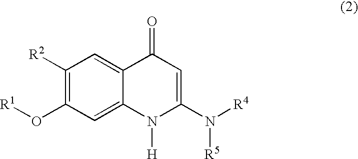

wherein R¹, R², R⁴ and R⁵ are as defined herein for compounds of formula (1); and the salts, solvates, hydrates, tautomers, isomers or N-oxides thereof.

Examples of aliphatic groups, represented by R¹ include C₁₋₆ alkyl groups as herein described. More particular examples include C₁₋₃ alkyl groups, such as —CH₃, —CH₂CH₃, —CH₂CH₂CH₃ or —CH(CH₃)CH₃. Examples of cycloaliphatic groups which may represent R¹ include C₃₋₆ cycloalkyl groups, such as those described previously. Examples of cycloalkyl-alkyl-groups which may represent R¹ include C₁₋₃ alkyl groups (as described herein) where a terminal hydrogen atom is replaced by a C₃₋₆ cycloalkyl ring (as described herein), for example, cyclopropylCH₂—.

In one group of compounds of formulae (1) or (2) R¹ is in particular a C₁₋₆ alkyl group. Especially preferred is when R¹ is a C₁₋₃ alkyl group. Most especially preferred is when R¹ is a methyl group.

In another group of compounds of formulae (1) or (2) R¹ is in particular a haloalkyl group. Especially preferred is when R¹ is a —CHF₂ or —CH₂F group.

One group of compounds has the formulae (1) or (2) wherein R² is a —CN group.

Another group of compounds of the invention has the formulae (1) or (2) wherein R² is an optionally substituted heteroaromatic group. In particular R² is an optionally substituted monocyclic ring heteroaromatic, especially a five-membered heteroaromatic group containing one, two, three or four heteroatoms selected from oxygen, sulfur or nitrogen atoms. Particular heteroaromatic groups which may represent R² include optionally substituted pyrrolyl, furyl, thienyl, imidazolyl, N—C₁₋₆alkylimidazolyl, oxazolyl, isoxazolyl, thiazolyl, isothiazolyl, ozadiazolyl, thiadiazolyl, triazolyl or pyrazolyl. Especially preferred is when R² is an oxazolyl group.

Particular examples of the group R⁴, in compounds of formulae (1) or (2), include -Alk¹-L¹-Alk²-R⁹, -Alk¹-L¹—R⁹, -Alk¹-R⁹, —L¹-Alk²-R⁹, —L¹—R⁹ or —R⁹wherein Alk¹, L¹, Alk² and R⁹ are as herein defined. R⁴ in one group of compounds of formulae (1) or (2) is the chain -Alk¹-L¹—R⁹. R⁴ is preferably the chain -Alk¹-R⁹.

Alk¹, when present in compounds of formulae (1) or (2), is preferably an optionally substituted aliphatic chain, in particular a C₁₋₆ alkylene chain, especially an optionally substituted —CH₂—, —CH₂CH₂—, —CH₂CH₂CH₂—, —CH(CH₃)CH₂— or —CH₂CH(CH₃)— chain, most especially a C₁₋₃ alkylene chain such as a —CH₂—, —CH₂CH₂— or —CH₂CH₂CH₂— chain.

Particular examples of L¹, when present in compounds of formulae (1) or (2), include —O— or —S— atoms or —C(O)—, —C(S)—, —S(O)—, —S(O))₂—, —C(O)O—, —OC(O)—, —N(R¹⁰)— [where R¹⁰ is as defined hereinbefore], —CON(R¹⁰)—, —CSN(R¹⁰)—, —N(R¹⁰)CO—, —N(R¹⁰)CS—, —S(O))₂N(R¹⁰)— or —N(R¹⁰)S(O))₂— groups. R¹⁰ is especially a hydrogen atom or a C₁₋₃ alkyl group, particularly a methyl group.

One group of compounds of the invention has the formulae (1) or (2) wherein Alk¹ is an optionally substituted aliphatic chain, L¹ and Alk² are each a covalent bond and R⁹ is a hydrogen atom. In compounds of this type Alk¹ is in particular an optionally substituted C₁₋₆ alkylene chain. In one particular group of compounds of this class R⁴ is especially a straight or branched C₁₋₆ alkyl group, particularly —CH₃, —CH₂CH₃, —CH(CH₃)₂, —(CH₂)₂CH₃ or —C(CH₃)₃. In another particular group of compounds of this class Alk¹ is a substituted C₁₋₆ alkylene chain, especially —CH₂—, —CH₂CH₂—, —(CH₂)₂CH₂—, —(CH₂)₃CH₂— or —CH₂C(CH₃)₂—.

Particular substituents present on the groups Alk¹ or Alk² include —CO₂H, —CO₂R¹⁶ [where R¹⁶ is as herein defined] —CONHR¹⁶, —CON(R¹⁶)₂, —COR ¹⁶, C₁₋₆ alkoxy, particularly methoxy or ethoxy; haloC₁₋₆alkoxy, particularly trifluoromethoxy or difluoromethoxy; —S(O)R¹⁶, —S(O)₂R¹⁶, amino, —NHR¹⁶ or —N(R¹⁶)₂ groups. R¹⁶ is in particular a C₁₋₃ alkyl group.

Another group of compounds of the invention has the formulae (1) or (2) wherein Alk¹ is an optionally substituted aliphatic chain, L¹ and Alk² are each a covalent bond and R⁹ is an optionally substituted cycloaliphatic, heterocycloaliphatic, aromatic or heteroaromatic group. Particular compounds of this type are those wherein R⁹ is an optionally substituted heterocycloaliphatic, aromatic or heteroaromatic group. Particular R⁹ examples include optionally substituted azetidinyl, pyrrolidinyl, pyrrolidinonyl, piperidinyl, imidazolidinyl, thiazolidinyl, piperazinyl, N—C₁₋₆ alkylpiperazinyl, especially N-methylpiperazinyl, N—C₁₋₆alkylpyrrolidinyl, especially N-methyl pyrrolidinyl, N—C₁₋₆ alkylpiperidinyl, especially N-methylpiperidinyl, homopiperazinyl, morpholinyl, thiomorpholinyl, oxazolidinyl, tetrahydrofuranyl, tetrahydropyranyl, phenyl, pyrrolyl, furyl, thienyl, imidazolyl, N—C₁₋₆ alkylimidazolyl, oxazolyl, isoxazolyl, thiazolyl, isothiazolyl, pyrazolyl, triazolyl, oxadiazolyl, thiadiazolyl, pyridyl, pyrimidinyl, pyridazinyl, pyrazinyl, tetrazolyl or triazinyl. In this group of compounds Alk¹ is in particular a C₁₋₃ alkylene chain, especially —CH₂— or —CH₂CH₂—. R⁹ in general in these compounds is especially an optionally substituted aromatic or heteroaromatic group. In another particular group of compounds of this type R⁹ is an optionally substituted cycloaliphatic group especially a C₃₋₆ cycloalkyl group.

A further group of compounds of the invention has the formulae (1) or (2) wherein Alk¹, Alk² and L¹ are each a covalent bond and R⁹ is an optionally substituted cycloaliphatic, heterocycloaliphatic, aromatic or heteroaromatic group. One preferred group of compounds is where R⁹ is an optionally substituted phenyl or monocyclic heteroaromatic group. In compounds of this type R⁹ is in particular an optionally substituted phenyl, pyridyl, pyrimidinyl, pyridazinyl or pyrazinyl group especially an optionally substituted phenyl or pyridyl group. R⁹ in one group of compounds is a phenyl or pyridyl group.

R⁵ in compounds of the invention is especially a hydrogen atom or a methyl group, particularly a hydrogen atom.

Another useful group of compounds of the invention has the formulae (1) or (2) wherein NR⁴R⁵ forms an optionally substituted heterocycloaliphatic group. In compounds of this type NR⁴R⁵ is in particular an optionally substituted azetidinyl or optionally substituted pyrrolidinyl, piperidinyl, piperazinyl, N—C₁₋₆alkylpiperazinyl, homopiperazinyl, morpholinyl or thiomorpholinyl group, especially a morpholinyl group. NR⁴R⁵ is also in particular an optionally substituted pyrrolidinyl or piperidinyl group. These groups may be fused to an optionally substituted monocyclic C₆₋₁₂aromatic group, such as phenyl or an optionally substituted monocyclic C₁₉heteroaromatic group containing for example one, two, three or four heteroatoms selected from oxygen, sulfur or nitrogen atoms. In particular NR⁴R⁵ is fused to an optionally substituted phenyl or five or six membered heteroaryl group. Particular examples of heteroaryl groups include pyrrolyl, furyl, thienyl, imidazolyl, N—C₁₋₆alkylimidazolyl, oxazolyl, isoxazolyl, thiazolyl, isothiazolyl, pyrazolyl, triazolyl, oxadiazolyl, thiadiazolyl, pyridyl, pyrimidinyl, pyridazinyl, pyrazinyl, especially pyridyl, pyrimidinyl or pyridazinyl. In one particular group of compounds NR⁴R⁵ is fused to an optionally substituted phenyl group and may in particular be an optionally substituted 2,3-dihydro-1H-indolyl, 2,3-dihydro-1H-isoindolyl, 1,2,3,4 tetrahydroquinolinyl or 1,2,3,4 tetrahydroisoquinolinyl group.

One group of optional substituents which may be present on aromatic or heteroaromatic groups in compounds of formulae (1) or (2) and in particular in R⁹ aromatic or heteroaromatic groups or in the aryl or heteroaryl groups optionally fused to NR⁴R⁵ include one, two, three or more atoms or groups selected from fluorine, chlorine, bromine, straight or branched C₁₋₆ alkyl, methoxy, OCF₃, OCF₂H, CF₃, CN, NHCH₃, N(CH₃)₂, CONH₂, CONHCH₃, CON(CH₃)₂, CO₂CH₃, CO₂CH₂CH₃, —CO₂C(CH₃)₃, or —COCH₃, —NHCOCH₃, —N(CH₃)COCH₃ or CO₂H or optionally substituted morpholinyl, thiomorpholinyl, piperazinyl, pyrrolidinyl, piperidinyl wherein the optional substituent are as herein defined.

One group of optional substituents which may be present on cycloaliphatic or heterocycloaliphatic groups in compounds of formulae (1) or (2) and in particular on the groups R⁹ or NR⁴R⁵, are one, two, three or more groups selected from C₁₋₃ alkoxy, OCF₃, OCF₂H, CF₃, C₁₋₃ alkylthio, —CN, NHCH₃, N(CH₃)₂, CONH₂, CONHCH₃, CON(CH₃)₂, CO₂CH₃, CO₂CH₂CH₃, —CO₂C(CH₃)₃, —COCH₃, —NHCOCH₃, —N(CH₃)COCH₃, CO₂H, or optionally substituted straight or branched C₁₋₃ alkyl, wherein the optional alkyl substituent is in particular —CN, C₁₋₃ alkoxy, NHCH₃, N(CH₃)₂, CONH₂, CONHCH₃, CON(CH₃)₂, CO₂CH₃, CO₂CH₂CH₃, —CO₂C(CH₃)₃, —COCH₃, —NHCOCH₃, —N(CH₃)COCH₃ or CO₂H.

When compounds of formulae (1) or (2) contain a heterocycloaliphatic or heteroaryl group having an available N atom this may in particular be substituted with an optionally substituted straight or branched C₁₋₃ alkyl group, especially a methyl group.

Particular compounds of the invention include:

2-(2,3-Dihydroindol-1-yl)-7-methoxy-6-oxazol-5-yl-1H-quinolin-4-one;

2-(Dihydro-1H-isoquinolin-2-yl)-7-methoxy-6-oxazol-5-yl-1H-quinolin-4-one;

2-(1,3-Dihydroisoindol-2-yl)-7-methoxy-6-oxazol-5-yl-1H-quinolin-4-one;

2-(6,7-Dimethoxy-3,4-dihydro-1H-isoquinolin-2-yl)-7-methoxy-6-oxazol-5-yl-1H-quinolin-4-one;

2-(5-Bromo-2,3-dihydroindol-1-yl)-7-methoxy-6-oxazol-5-yl-1H-quinolin-4-one;

7-Methoxy-2-(2-methyl-2,3-dihydroindol-1-yl)-6-oxazol-5-yl-1H-quinolin-4-one;

7′-Methoxy-6′-oxazol-5-yl-3,4-dihydro-2H,1′H-[1,2′]biquinolinyl-4′-one;

and the salts, solvates, hydrates, tautomers, isomers or N-oxides thereof.

Compounds of formulae (1) or (2) are potent inhibitors of IMPDH. The ability of the compounds to act in this way may be simply determined by employing tests such as those described in the Examples hereinafter.

Thus the compounds of the invention may be used in the treatment of IMPDH-associated disorders. The invention extends to such a use and in general to the use of the compounds of formulae (1) or (2) for the manufacture of a medicament for treating such diseases and disorders.

“IMPDH-associated disorders” refers to any disorder or disease state in which inhibition of the enzyme IMPDH (inosine monphosphate dehydrogenase, EC1.1.1.205, of which there are presently two known isozymes referred to as IMPDH type 1 and IMPDH type 2) would modulate the activity of cells (such as lymphocytes or other cells) and thereby ameliorate or reduce the symptoms or modify the underlying cause(s) of that disorder or disease. There may or may not be present in the disorder or disease an abnormality associated directly with the IMPDH enzyme. Examples of IMPDH-associated disorders include transplant rejection and autoimmune disorders, such as rheumatoid arthritis, lupus, multiple sclerosis, juvenile diabetes, asthma, and inflammatory bowel disease, as well as inflammatory disorders, cancer and tumors, T-cell mediated hypersensitivity diseases, ischemic or reperfusion injury, viral replication diseases, proliferative disorders and vascular diseases.

Use of the compounds of the present invention is exemplified by, but is not limited to, treating a range of disorders such as: treatment of transplant rejection (e.g. kidney, liver, heart, lung, pancreas (e.g., islet cells), bone marrow, cornea, small bowel, skin allografts, skin homografts (such as employed in burn treatment), heart valve xenografts, serum sickness, and graft vs. host disease, in the treatment of autoimmune diseases, such as rheumatoid arthritis, psoriatic arthritis, multiple sclerosis, juvenile diabetes, asthma, inflammatory bowel disease (such as Crohn's disease and ulcerative colitus), pyoderma gangrenum, lupus (systemic lupus erythematosis), myasthenia gravis, psoriasis, eczema, dermatitis, dermatomyosis, atopic dermatitis; multiple sclerosis, seborrhoea, pulmonary inflammation, eye uveitis, hepatitis, Grave's disease, Hashimoto's thyroiditis, autoimmune thyroiditis, Behcet's or Sjorgen's syndrome (dry eyes/mouth), pernicious or immunohaemolytic anaemia, Addison's disease (autoimmune disease of the adrenal glands), idiopathic adrenal insufficiency, autoimmune polyglandular disease (also known as autoimmune polyglandular syndrome) glomerulonephritis, scleroderma, morphea, lichen planus, viteligo (depigmentation of the skin), alopecia areata, autoimmune alopecia, autoimmune hypopituatarism, cicatricial pemphigoid, Gullivan-Barre syndrome, and alveolitis; in the treatment of T-cell mediated hypersensitivity diseases, including contact hypersensitivity, delayed-type hypersensitivity, contact dermatitis (including that due to poison ivy), urticaria, skin allergies, respiratory allergies (hayfever, allergic rhinitis) and gluten-sensitive enteropathy (Celiac disease); in the treatment of inflammatory diseases such as osteoarthritis, acute pancreatitis, chronic pancreatitis, asthma, acute respiratory distress syndrome, Sezary's syndrome and vascular diseases which have an inflammatory and or a proliferatory component such as restenosis, stenosis and artherosclerosis; in the treatment of cancer and tumor disorders, such as solid tumors, lymphomas and leukemia; in the treatment of fungal infections such as mycosis fungoides; in protection from ischemic or reperfusion injury such as ischemic or reperfusion injury that may have been incurred during organ transplantation, myocardial infarction, stroke or other causes; in the treatment of DNA or RNA viral replication diseases, such as herpes simplex type 1 (HSV-1), herpes simplex type 2 (HSV-2), hepatitis (including hepatitis B and hepatitis C) cytomegalovirus, Epstein-Barr, human immundeficiency virus (HIV) and influenza.

Additionally, IMPDH is also known to be present in bacteria and thus may regulate bacterial growth. As such, the IMPDH-inhibitor compounds of the present invention may be useful in treatment or prevention of bacterial infection, alone or in combination with other antibiotic agents.

In a particular embodiment, the compounds of the present invention are useful for the treatment of the afore mentioned exemplary disorders irrespective of their etiology, for example, for the treatment of lupus, psoriasis, inflammatory bowl disease, multiple sclerosis, atopic dermatitis or rheumatoid arthritis.

In another particular embodiment the compounds of the present invention are of particular use for the treatment of DNA or RNA viral replication diseases, such as hepatitis (including hepatitis B and hepatitis C) cytomegalovirus, human immundeficiency virus (HIV) and influenza.

In an additional particular embodiment the compounds of the present invention are of particular use for the treatment of cancer and tumour disorders, such as solid tumors, lymphoma, leukemia and other forms of cancer.

The compounds of formulae (1) or (2) can be used alone or in combination with other therapeutic or prophylactic agents, such as anti-virals, anti-inflammatory agents, antibiotics and immunosuppressants for the treatment or prophylaxis of transplant rejection and autoimmune disease.

For the prophylaxis or treatment of disease the compounds according to the invention may be administered as pharmaceutical compositions, and according to a further aspect of the invention we provide a pharmaceutical composition which comprises a compound of formulae (1) or (2) together with one or more pharmaceutically acceptable carriers, excipients or diluents.

Alternate compositions of this invention comprise a compound formula (1) or a salt thereof; an additional agent selected from an immunosuppressant, an anti-cancer agent, an anti-viral agent, anti-inflammatory agent, anti-fungal agent, anti-vascular hyperproliferation agent or an antibiotic agent; and any pharmaceutically acceptable carrier, adjuvant or vehicle.

Thus, for example, additional immunosuppression agents include, but are not limited to, cyclosporin A, FK506, rapamycin, leflunomide, deoxyspergualin, prednisone, azathioprine, OKT3, ATAG, interferon and mizoribine. Additional anti-vascular hyperproliferative agents include, but are not limited to, HMG Co-A reductase inhibitors such as lovastatin, thromboxane A2 synthetase inhibitors, ciprostene, trapidil, eicosapentanoic acid, ACE inhibitors, low molecular weight heparin, and rapamycin. Additional anti-cancer agents include, but are not limited to, cis-platin, actinomycin D, amsacrine, mitoxantrone, doxorubicin, vincristine, vinblastine, etoposide, tenipaside, taxol, colchicine, cyclosporin A, phenothiazines, interferon and thioxantheres. Additional anti-viral agents include, but are not limited to, Cytovene, Ganiclovir, trisodium phosphonoformate, Ribavirin, d4T, ddl, AZT and acyclovir.

The above other therapeutic agents, when employed in combination with the compounds of the present invention, may be used, for example, in amounts generally indicated for use in standard formularies (e.g. in the Physician's Desk Reference (PDR)) or as determined using routine pharmaceutical dosing methods.

Pharmaceutical compositions according to the invention may take a form suitable for oral, buccal, parenteral, nasal, topical, vaginal or rectal administration, or a form suitable for administration by inhalation or insufflation.

For oral administration, the pharmaceutical compositions may take the form of, for example, tablets, lozenges or capsules prepared by conventional means with pharmaceutically acceptable excipients such as binding agents (e.g. pregelatinised maize starch, polyvinylpyrrolidone or hydroxypropyl methylcellulose); fillers (e.g. lactose, microcrystalline cellulose or calcium hydrogen phosphate); lubricants (e.g. magnesium stearate, talc or silica); disintegrants (e.g. potato starch or sodium glycollate); or wetting agents (e.g. sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents, emulsifying agents, non-aqueous vehicles and preservatives. The preparations may also contain buffer salts, flavouring, colouring and sweetening agents as appropriate.

Preparations for oral administration may be suitably formulated to give controlled release of the active compound

For buccal administration the compositions may take the form of tablets or lozenges formulated in conventional manner.

The compounds for formulae (1) or (2) may be formulated for parenteral administration by injection e.g. by bolus injection or infusion. Formulations for injection may be presented in unit dosage form, e.g. in glass ampoule or multi dose containers, e.g. glass vials. The compositions for injection may take such forms as suspensions, solutions or emulsions in oily or aqueous vehicles, and may contain formulatory agents such as suspending, stabilising, preserving and/or dispersing agents. Alternatively, the active ingredient may be in powder form for constitution with a suitable vehicle, e.g. sterile pyrogen-free water, before use. For particle mediated administration the compounds of formulae (1) or (2) may be coated on particles such as microscopic gold particles.

In addition to the formulations described above, the compounds of formulae (1) or (2) may also be formulated as a depot preparation. Such long acting formulations may be administered by implantation or by intramuscular injection.

For nasal administration or administration by inhalation, the compounds for use according to the present invention are conveniently delivered in the form of an aerosol spray presentation for pressurised packs or a nebuliser, with the use of suitable propellant, e.g. dichlorodifluoromethane, trichlorofluoromethane, dichlorotetrafluoroethane, carbon dioxide or other suitable gas or mixture of gases.

For vaginal or rectal administration the compounds of formulae (1) or (2) may be formulated as a suppository. These formulations may be prepared by mixing the active ingredient with a suitable non-irritating excipient which is a solid at room temperature but liquid at the body temperature. Such materials include for example cocoa butter and polyethylene glycols.

The compositions may, if desired, be presented in a pack or dispenser device which may contain one or more unit dosage forms containing the active ingredient. The pack or dispensing device may be accompanied by instructions for administration.

The quantity of a compound of the invention required for the prophylaxis or treatment of a particular condition will vary depending on the compound chosen, and the condition of the patient to be treated. In general, however, daily dosages may range from around 100 ng/kg to 100 mg/kg e.g. around 0.01 mg/kg to 40 mg/kg body weight for oral or buccal administration, from around 10 ng/kg to 50 mg/kg body weight for parenteral administration and around 0.05 mg to around 1000 mg e.g. around 0.5 mg to around 1000 mg for nasal administration or administration by inhalation or insufflation.

The compounds of the invention may be prepared by a number of processes as generally described below and more specifically in the Examples hereinafter. Many of the reactions described are well-known standard synthetic methods which may be applied to a variety of compounds and as such can be used not only to generate compounds of the invention, but also where necessary the intermediates thereto.

In the following process description, the symbols R¹-R⁵ when used in the formulae depicted are to be understood to represent those groups described above in relation to formulae (1) or (2) unless otherwise indicated. In the reactions described below, it may be necessary to protect reactive functional groups, for example hydroxy, amino, thio or carboxy groups, where these are desired in the final product, to avoid their unwanted participation in the reactions. Conventional protecting groups may be used in accordance with standard practice [see, for example, Green, T. W. in “Protective Groups in Organic Synthesis”, John Wiley and Sons, (1999) and the examples herein]. In some instances, deprotection may be the final step in the synthesis of a compound of formulae (1) or (2) and the processes according to the invention described hereinafter are to be understood to extend to such removal of protecting groups.

For example, a compound of formulae (1) or (2) where X is an O atom and R³ is a hydrogen atom may be prepared by following the general route as shown in Scheme A:

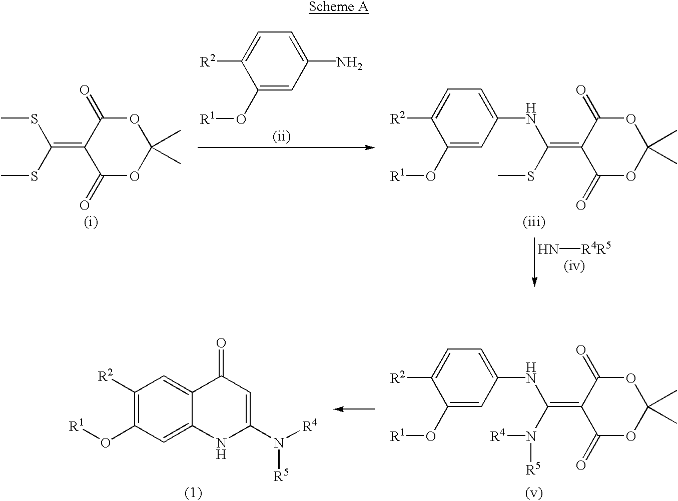

A quinolone of general formula (1) may be prepared using similar methodology to that reported by Bang-Chi et al (Synthesis, pp. 317-320, (1989)) Thus commercially available 5-(bismethylsulfanylmethylene)-2,2-dimethyl-[1,3]dioxane-4,6-dione (i) may be treated with an amine of general formula (ii) to give a malonate of general formula (iii). Appropriate conditions for this reaction may involve heating in an alcoholic solvent e.g. ethanol at reflux temperature for a suitable period of time e.g. 2 hours. The malonate (iii) may then be treated with an amine of general formula (iv) using appropriate conditions, for example, in the presence of mercury (II) chloride at room temperature or with heating, to afford a compound of general formula (v). The reaction may be performed without solvent (for example, if one of the reagents is a liquid) or in the presence of a small amount of a suitable solvent e.g. tetrahydrofuran, DMF or diphenyl ether. The compound of formula (v) may then be cyclised, for example, by heating in a solvent such as diphenyl ether at the reflux temperature in to afford a quinolone of formula (1 ) wherein R³ is a hydrogen atom. The cyclisation may also be performed in a microwave reactor in for example diphenyl ether in the presence of a co-solvent such as N-methylpyrrolidinone. Alternatively the compound of formula (iii) may be converted to a compound of formula (1) in a one-pot reaction without the need to isolate a compound of formula (v) using similar methodology as described above.

Alternatively when R³ in compounds of formula (1) is a —CN group, compounds of this type may be prepared in a similar manner to the general route described for Scheme A. See also Tominaga et al J. of Heterocyclic Chem. 27, (5), pp.1217-1225, (1990). Thus instead of using the compound of formula (i) commercially available 2-cyano-3,3-bis-methylsulfanylacrylic acid methyl ester (vi):

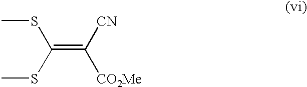

may be reacted with an amine of general formula (ii) employing the same methodology as described in Scheme A. The intermediate thus formed may be further manipulated using the same methods as described above and an amine of formula (iv) to afford a compound of formula (1) wherein R³ is a —CN group.

When R³ in compounds of formula (1) is a —CO₂H, —CO₂R⁶ or —CONR⁷R⁸ group such compounds may be prepared from the corresponding compound of formula (1) where R³ is a —CN group using standard conditions known to those skilled in the art. Thus, nitrile (CN) groups may be hydrolysed in the presence of acid or base to give an acid or primary amide using standard methods. The groups thus formed may then be further functionalised using standard alkylation and esterification techniques.

Compounds of formula (1) in which X is an O atom may be converted to their thioketone analogues using standard techniques, for example, by reaction with Lawesson's reagent in a suitable solvent, such as tetrahydrofuran or toluene.

Intermediates of formulae (ii) and (iv) and any other intermediates required to obtain compounds of formulae (1) and (2), if not available commercially, may be prepared by methods known to those skilled in the art following procedures set forth in references such as Rodd's Chemistry of Carbon Compounds, Volumes 1-15 and Supplementals (Elsevier Science Publishers, 1989), Fieser and Fieser's Reagents for Organic Synthesis, Volumes 1-19 (John Wiley and Sons, 1999), Comprehensive Heterocyclic Chemistry, Ed. Katritzky et al, Volumes 1-8, 1984 and Volumes 1-11, 1994 (Pergamon), Comprehensive Organic Functional Group Transformations, Ed. Katritzky et al, Volumes 1-7, 1995 Pergamon), Comprehensive Organic Synthesis, Ed. Trost and Flemming, Volumes 1-9, (Pergamon, 1991), Encyclopaedia of Reagents for Organic Synthesis Ed. Paquette, Volumes 1-8 (John Wiley and Sons, 1995), Larock's Comprehensive Organic Transformations (VCH Publishers Inc., 1989) and March's Advanced Organic Chemistry (John Wiley and Sons, 1992).

Thus amines of general formula (ii) may be prepared in a variety of ways. For example, the compound of formula (iii) where R¹ is a methyl group and R² is an oxazole group may be prepared using methods known in the literature (CAS 198821-79-3).

Alternatively amines of formula (ii), where R² is an optionally substituted heteroaromatic group, may be prepared using the route as shown in Scheme B:

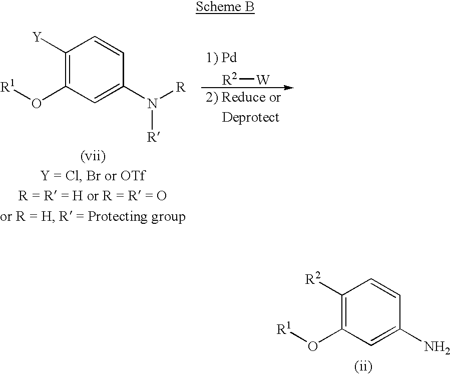

For example, a compound of formula (vii), where Y is a halogen atom e.g. Cl or Br or a suitable leaving group e.g. trifluoromethylsulfonyloxy (OTf) and —NRR′ is a nitro group or an amine group (which may be suitably protected), may be reacted with a derivative of the desired heteroaromatic group (R²—W, where W is as described below) utilising a palladium catalysed cross coupling reaction. The following literature methodology may be used to perform this coupling reaction according to the nature of the W group; e.g. when W is a hydrogen atom (Heterocycles, 31, pp. 1951-1958, (1990)); the zinc species (W=ZnCl) (J Organomet. Chem., 390, pp. 389-398, (1990); Tetrahedron, 53, pp. 7237-7254, (1997)); the mercury species (W=HgBr) (Chem. Heterocycl. Compd., 19, pp. 1159-1162, (1983)) or a boron derivative (W=B(OH)₂, W=BEt₂) (J. Med. Chem., 40, pp. 3542-3550, (1997); J. Org. Chem., 63, pp. 8295-8303, (1998)). The resulting coupled product may require further manipulation, depending on the nature of the —NRR′ group, in order to obtain an amine of formula (ii). For example, when —NRR′ is a nitro group this may be reduced to an amine using standard techniques, or when —NRR′ is a protected amine the protecting group may be removed using standard methodology. It will be appreciated that the various R²—W derivatives are either commercially available or may be prepared using methods known to those skilled in the art. In a similar manner the compounds of formula (vii) are either commercially available or may be prepared using methods known to those skilled in the art. For example, the compound of formula (vii) may be prepared by alkylation of the phenol precursor of (vii) using standard techniques.

When R² in compounds of formula (1) is a —CN group, these may be prepared using similar methodology to that described herein starting from a compound of formula (viii):

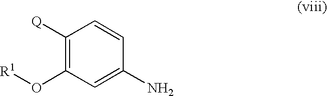

wherein Q is a halogen atom e.g. bromine or a protected phenol e.g. tert-butyldimethylsilyloxy group. Thus an amine of formula (viii) may be used instead of the amine of formula (ii) in the general route as shown in Scheme A. The quinolone thus formed may then be further converted using methods known to those skilled in the art to give a compound of formula (1) wherein R² is a CN group. For example, when Q is a bromine atom this may be reacted with a cyanide group e.g. zinc cyanide in the presence of a palladium catalyst e.g. tetrakis(triphenylphosphine)palladium (0) in for example N,N-dimethylformamide at 100° C. Alternatively when Q is a protected phenol group this may, after deprotection, be converted into a leaving group e.g. trifluoromethylsulfonyloxy and displaced in a similar manner to that as described above for the bromide.

An amine of formula (viii) may be prepared using standard methods known to those skilled in the art. For example when Q is a bromine atom this may be prepared using the general route as shown in Scheme C:

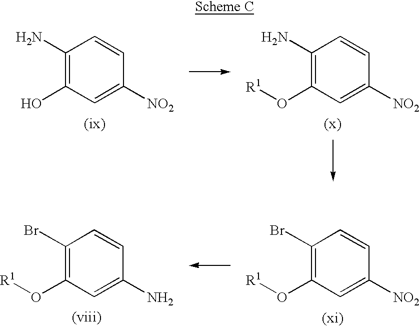

Thus the commercially available compound of formula (ix) may be alkylated e.g. using a reagent R¹Y (where Y is as defined earlier) in the presence of a base, at the phenol position using standard methodology to give a compound of formula (x). The compound of formula (x) may then be converted to a bromide of formula (xi) using methods known to those skilled in the art, for example by treatment with sodium nitrite in the presence of aqueous hydrogen bromide followed by the addition of copper bromide and hydrogen bromide. The compound of formula (viii) may then be prepared by reduction of the nitro group in the compound of formula (xi) using for example palladium catalysed hydrogenation.

It will be appreciated that compounds of formula (1) or any preceding intermediates such as intermediates of formula (iv) may be further derivatised by one or more standard synthetic methods employing substitution, oxidation, reduction or cleavage reactions. Particular substitution approaches include conventional alkylation, arylation, heteroarylation, acylation, thioacylation, halogenation, sulphonylation, nitration, formylation and coupling procedures. It will be appreciated that these methods may also be used to obtain or modify other compounds of any of formula (1) or any preceding intermediates where appropriate functional groups exist in these compounds.

For example, ester groups such as —CO₂R¹¹ or —CO₂Alk⁴ in the compounds may be converted to the corresponding acid [—CO₂H] by acid- or base-catalysed hydrolysis depending on the nature of the groups R¹¹ or Alk⁴. Acid- or base-catalysed hydrolysis may be achieved for example by treatment with an organic or inorganic acid, e.g. trifluoroacetic acid in an aqueous solvent or a mineral acid such as hydrochloric acid in a solvent such as dioxan or an alkali metal hydroxide, e.g. lithium hydroxide in an aqueous alcohol, e.g. aqueous methanol. Similarly an acid [—CO₂H] may be prepared by hydrolysis of the corresponding nitrile [—CN], using for example a base such as sodium hydroxide in a refluxing alcoholic solvent, such as ethanol.

In another example, —OH groups may be generated from the corresponding ester [e.g. CO₂Alk⁴ or CO₂R¹¹] or aldehyde [—CHO] by reduction, using for example a complex metal hydride such as lithium aluminium hydride in diethyl ether or tetrahydrofuran or sodium borohydride in a solvent such as methanol. Alternatively an alcohol may be prepared by reduction of the corresponding acid [—CO₂H], using for example lithium aluminium hydride in a solvent such as tetrahydrofuran.

Alcohol groups may be converted into leaving groups, such as halogen atoms or sulfonyloxy groups such as an alkylsulfonyloxy, e.g. trifluoromethylsulfonyloxy or arylsulfonyloxy, e.g. p-toluenesulfonyloxy group using conditions known to those skilled in the art. For example, an alcohol may be reacted with thionyl chloride in a halogenated hydrocarbon e.g., dichloromethane to yield the corresponding chloride. A base e.g., triethylamine may also be used in the reaction.

In another example, alcohol or phenol groups may be converted to ether groups by coupling a phenol with an alcohol in a solvent such as tetrahydrofuran in the presence of a phosphine, e.g. triphenylphosphine and an activator such as diethyl-, diisopropyl-, or dimethylazodicarboxylate. Alternatively ether groups may be prepared by deprotonation of an alcohol, using a suitable base e.g. sodium hydride followed by subsequent addition of an alkylating agent, such as an alkylhalide.

Aldehyde [—CHO] groups may be obtained by oxidation of a corresponding alcohol using well-known conditions. For example using an oxidising agent such as a periodinane e.g. Dess Martin, in a solvent such as a halogenated hydrocarbon, e.g. dichloromethane. An alternative oxidation may be suitably activating dimethyl sulfoxide using for example, oxalyl chloride, followed by addition of an alcohol, and subsequent quenching of the reaction by the addition of an amine base, such as triethylamine. Suitable conditions for this reaction may be using an appropriate solvent, for example, a halogenated hydrocarbon, e.g. dichloromethane at −78° C. followed by subsequent warming to room temperature.

In a further example primary amine (—NH₂) or secondary amine (—NH—) groups may be alkylated using a reductive alkylation process employing an aldehyde and a borohydride, for example sodium triacetoxyborohyride or sodium cyanoborohydride, in a solvent such as a halogenated hydrocarbon, e.g. dichloromethane, a ketone such as acetone, or an alcohol, e.g. ethanol, where necessary in the presence of an acid such as acetic acid at around ambient temperature.

In a further example, amine [—NH₂] groups may be obtained by hydrolysis from a corresponding imide by reaction with hydrazine in a solvent such as an alcohol, e.g. ethanol at ambient temperature.

In another example, a nitro [—NO₂] group may be reduced to an amine [—NH₂], for example by catalytic hydrogenation using for example hydrogen in the presence of a metal catalyst, for example palladium on a support such as carbon in a solvent such as an ether, e.g. tetrahydrofuran or an alcohol e.g. methanol, or by chemical reduction using for example a metal, e.g. tin or iron, in the presence of an acid such as hydrochloric acid.

In a further example amine (—CH₂NH₂) groups may be obtained by reduction of nitrites (—CN), for example by catalytic hydrogenation using for example hydrogen in the presence of a metal catalyst, for example palladium on a support such as carbon, or Raney nickel, in a solvent such as an ether e.g. a cyclic an ether, e.g. a cyclic ether such as tetrahydrofuran, at a temperature from −78° C. to the reflux temperature.

Aromatic halogen substituents in the compounds may be subjected to halogen-metal exchange by treatment with a base, for example a lithium base such as n-butyl or t-butyl lithium, optionally at a low temperature, e.g. around −78° C., in a solvent such as tetrahydrofuran and then quenched with an electrophile to introduce a desired substituent. Thus, for example, a formyl group may be introduced by using dimethylformamide as the electrophile; a thiomethyl group may be introduced by using dimethyldisulphide as the electrophile. Aromatic halogen substituents may also be subjected to palladium catalysed reactions, to introduce, for example, acid, ester, cyano or amino substituents.

In another example, sulfur atoms in the compounds, for example when present in a linker group L¹ or L² may be oxidised to the corresponding sulphoxide or sulphone using an oxidising agent such as a peroxy acid, e.g. 3-chloroperoxybenzoic acid, in an inert solvent such as a halogenated hydrocarbon, e.g. dichloromethane, at around ambient temperature.

N-oxides of compounds of formulae (1) or (2) may be prepared for example by oxidation of the corresponding nitrogen base using an oxidising agent such as hydrogen peroxide in the presence of an acid such as acetic acid, at an elevated temperature, for example around 70° C. to 80° C., or alternatively by reaction with a peracid such as peracetic acid in a solvent, e.g. dichloromethane, at ambient temperature.

Salts of compounds of formulae (1) or (2) may be prepared by reaction of a compound of formulae (1) or (2) with an appropriate base or acid in a suitable solvent or mixture of solvents e.g. an organic solvent such as an ether e.g. diethylether, or an alcohol, e.g. ethanol or an aqueous solvent using conventional procedures. Salts of compounds of formulae (1) or (2) may be exchanged for other salts by use of conventional ion-exchange chromatography procedures.

Where it is desired to obtain a particular enantiomer of a compound of formulae (1) or (2) this may be produced from a corresponding mixture of enantiomers using any suitable conventional procedure for resolving enantiomers.

Thus for example diastereomeric derivatives, e.g. salts, may be produced by reaction of a mixture of enantiomers of formulae (1) or (2) e.g. a racemate, and an appropriate chiral compound, e.g. a chiral base. The diastereomers may then be separated by any convenient means, for example by crystallisation and the desired enantiomer recovered, e.g. by treatment with an acid in the instance where the diastereomer is a salt.

In another resolution process a racemate of formulae (1) or (2) may be separated using chiral High Performance Liquid Chromatography. Alternatively, if desired a particular enantiomer may be obtained by using an appropriate chiral intermediate in one of the processes described above.

Chromatography, recrystallisation and other conventional separation procedures may also be used with intermediates or final products where it is desired to obtain a particular geometric isomer of the invention.

The following Examples illustrate the invention. All temperatures are in ° C. Where experimental detail is not given for the preparation of a reagent it is either commercially available, or it is known in the literature, for which the CAS number is quoted. The compounds are named with the aid of Beilstein Autonom supplied by MDL Information Systems GmbH, Theodor-Heuss-Allee 108, D-60486 Frankfurt, Germany.

¹H NMR spectra were obtained at 300 MHz or 400 MHz unless otherwise indicated.

LCMS conditions:
HP1100 (Diode Array) linked to a Finnigan LC-Q Mass Spectrometer, ESI
mode with Pos/Neg ionization

| | |
|---|---|
| Column: | Luna C18(2) 100 × 4.6 mm, 5 μm particle size Analytical column |
| Column Temp: | Not controlled |
| Mobile Phase: | A: Water + 0.08% formic acid |
| | B: Acetonitrile + 0.1% formic acid |
| Flow rate: | 2ml/min |
| Gradient: | Time (mins): % Composition B: |
| 5 | |
| 6.50 | 95 |
| 8.00 | 95 |
| 8.05 | 5 |
| Run time: | 10.00 mins |
| Typical Injection Vol: | 5 μl |
| Detector Wavelength: DAD | 205-330 nm |
| Preparative LC conditions: | |
| Gilson 215 liquid handler setup. | |
| Column: | Luna C18(2) 250 × 21.2 mm, 5 μn particle size PREP column |
| Column Temp: | Ambient |
| Gradient: | Variable - depends on retention of sample in LCMS |
| | screen |
| Run Time: | 20 mins |
| Flow rate: | 25 ml/min |
| Typical Injection Vol: | 750 μ1 of 25 mg/ml solution |
| Detector Wavelength: | 210 and 254 nm |
| Method A: | |
| Mobile Phase: | A: Water + 0.08% formic acid |
| | B: Acetonitrile + 0.1% formic acid |
| Method B: | |
| Mobile phase: | A: Water + 0.1% ammonia |
| | B: Acetonitrile + 0.1% ammonia |
| Method C: | |
| Mobile phase | A: 10 mM Ammonium acetate in water (pH 5.8) |
| | B: 10 mM Ammonium acetate in acetonitrile (pH 5.8) |

Abbreviations used:
Boc - tert-Butoxycarbonyl
CDCl₃ Deuterated chloroform
DCM Dichloromethane
DMF N,N-Dimethylformamide
d₆-DMSO Deuterated dimethylsulfoxide
EtOAc Ethyl acetate
MeOH Methanol
d4-MeOH - Deuterated methanol
THF Tetrahydrofuran

### Intermediate 1

### 5-[(Methoxy-4-oxazol-5-yl-phenylamino)methyl-sulfanylmethylene]-2,2-dimethyl[1,3]dioxane-4,6-dione

A mixture of 3-methoxy-4-oxazol-5-yl-aniline (CAS 198821-79-3) (0.95 g, 5 mmol) and 5-(bismethylsulfanylmethylene)-2,2-dimethyl-[1,3]dioxane-4,6-dione (1.24 g) in ethanol (10 ml) was stirred and heated at reflux for 2 hours. The solvent was removed in vacuo and the residue was purified by column chromatography on silica eluting with 50-100% ethyl acetate/hexane to yield the title compound as a yellow solid (1.61 g, 83%). TLC R_{f} 0.19 (50% EtOAc/hexane).

¹H-NMR 300 MHz (CDCl₃) 12.9-12.8 (1H, s, br), 7.95 (1H, s), 7.85 (1H, d), 7.60 (1H, s), 7.05 (1H, dd), 6.95 (1H, d), 4.00 (3H, s), 2.35 (3H, s), 1.78 (6H, s).

### Intermediate 2

### 5-[(3-Methoxy-4-oxazol-5-yl-phenylamino)phenylaminomethylene]-2,2-dimethyl[1,3]dioxane-4,6-dione

A mixture of Intermediate 1 (500 mg, 1.28 mmol), aniline (2 ml) and mercury (II) chloride (348 mg) were stirred at room temperature for 30 minutes. The mixture was diluted in dichloromethane (20 ml), filtered and washed with dichloromethane (5 ml). The solvent was removed in vacuo and the residue purified by column chromatography on silica eluting with 50% ethyl acetate/hexane to yield the title compound as a pale yellow solid (422 mg, 76%). TLC R_{f} 0.25 (50% EtOAc/hexane).

¹H-NMR 300 MHz (CDCl₃) 12.0-11.9 (2H, s, br), 7.85 (1H, s), 7.45 (1H, d), 7.40 (1H, s), 7.05 (2H, m), 6.92 (3H, m), 6.60 (1H, dd), 6.45 (1H, d), 3.78 (3H, s), 1.80 (6H, s).

### Intermediate 3

### 5-[(3-Methoxy-4-oxazol-5-yl-phenylamino)(pyridin-3-ylamino)methylene]-2,2-dimethyl[1,3]dioxane-4,6-dione

A mixture of Intermediate 1 (100 mg, 0.26 mmol), 3-aminopyridine (100 mg), mercury (II) chloride (70 mg), and anhydrous THF (0.5 ml) were stirred at 80° C. under an atmosphere of nitrogen. After 30 minutes the mixture was cooled to room temperature, diluted with dichloromethane (20 ml), filtered and washed with dichloromethane (5 ml). The solvent was removed in vacuo and the residue purified by preparative HPLC (method A) to yield the title compound as a pale yellow solid (70 mg, 63%). TLC R_{f} 0.46 (EtOAc). MS 437 [M+H]⁺. ¹H-NMR 300 MHz (d₆-DMSO) 11.5-11.4 (2H, d, br), 8.45 (1H, s), 8.2 (1H, d), 7.60 (1H, m), 7.55-7.50 (2H, m), 7.20 (1H, dd), 6.95 (1H, s), 6.90 (1H, d), 3.90 (3H, s), 1.80 (6H, s).

Intermediates 4-22 were prepared in a similar manner to Intermediate 3:

### Intermediate 4

### 5-[Amino-(3-methoxy-4-oxazol-5-yl-phenylamino)-methylene]-2,2-dimethyl[1,3]dioxane-4,6-dione

From Intermediate 1 (250 mg, 0.6 mmol), ammonium hydroxide (2 ml), mercury (II) chloride (174 mg, 0.64 mmol) to give the title compound (225 mg, 92%). TLC R_{f} 0.24 (50% EtOAc/heptane).

¹H-NMR 300 MHz (CDCl₃) 11.45 (1H, s, br), 9.65 (1H, s, br), 7.92 (1H, s), 7.88-7.86 (1H, d), 7.60 (1H, s), 7.03-6.98 (1H, dd), 6.86 (1H, s), 5.68 (1H, s, br), 4.00 (3H, s), 1.74 (6H, s).

### Intermediate 5

### 4-[(2,2-Dimethyl-4,6-dioxo[1,3]dioxan-5-ylidene)(3-methoxy-4-oxazol-5-yl-phenylamino)methyl]piperazine-1-carboxylic acid tert-butyl ester

From Intermediate 1 (440 mg, 1.12 mmol), tert-butyl piperazine carboxylate (420 mg, 2.25 mmol) and mercury (II) chloride (311 mg, 1.12 mmol) to afford the title compound as a yellow solid (578 mg, 98%). TLC R_{f} 0.35 (5% MeOH/DCM). MS 358 [M−H]⁻.

¹H-NMR 300 MHz (CDCl₃) 9.84 (1H, s, br), 7.93 (1H, s), 7.80-7.75 (1H, d), 7.56 (1H, s), 6.78-6.70 (2H, m), 4.0 (3H, s), 3.53-3.45 (4H, m), 3.30-3.25 (4H, m) 1.77(6H, s), 1.40 (9H, s).

### Intermediate 6

### 5-[-{3-Methoxy-4-oxazol-5-yl-phenylamino)[(pyridin-3-ylmethyl)amino]-methylene}-2,2-dimethyl[1,3]dioxane-4,6-dione

From Intermediate 1 (250 mg, 0.64 mmol), 3-aminomethylpyridine (207 mg, 1.92 mmol) and mercury (II) chloride (174 mg, 0.64 mmol). Purification by column chromatography on silica eluting with 10% methanol/dichloromethane afforded the title compound as an off-white solid (255 mg, 89%). MS 451 [M+H]⁺.

¹H-NMR 300 MHz (CDCl₃) 11.70 (1H, s), 10.60 (1H, s), 8.55 (1H, d), 8.25 (1H, s), 8.00 (1H, s), 7.80 (1H, d), 7.60 (1H, s), 7.40 (1H, d), 7.25 (1H, m), 6.90 (1H, d), 6.70 (1H, s), 4.20 (2H, d), 3.90 (3H, s), 1.80 (6H, s).

### Intermediate 7

### 5-{3-Methoxy-4-oxazol-5-yl-phenylamino)[(furan-2-ylmethyl)amino]-methylene}-2,2-dimethyl[1,3]dioxane-4,6-dione

From Intermediate 1 (250 mg, 0.64 mmol), fufurylamine (170 μl, 1.92 mmol) and mercury (II) chloride (174 mg, 0.64 mmol). Purification by column chromatography on silica eluting with 10% methanol/dichloromethane afforded the title compound as an off-white solid (250 mg, 89%). TLC R_{f} 0.62 (10% MeOH/DCM).

¹H-NMR 300 MHz (CDCl₃) 8.10 (1H, s), 8.75 (1H, d), 8.45 (1H, s), 8.35 (1H, d), 7.95 (1H, d), 7.85 (1H, dd), 6.20 (1H, m), 6.10 (1H, d), 4.10 (2H, s), 3.90 (3H, s), 1.60 (6H, s).

### Intermediate 8

### 5-[[(1-Ethylpyrrolidin-2-ylmethyl)amino]-(3-methoxy-4-oxazol-5-yl-phenylamino)methylene]-2,2-dimethyl[1,3]dioxane-4,6-dione

From Intermediate 1 (250 mg, 0.64 mmol), N-ethyl-2-aminomethylpyrrolidine (280 μl, 1.03 mmol) and mercury (II) chloride (174 mg, 0.64 mmol). Purification by column chromatography on silica eluting with 5% methanol/dichloromethane yielded the title compound as an off-white solid (292 mg, 97%). TLC R_{f} 0.52 (10% MeOH/DCM). MS 471 [M+H]⁺.

### Intermediate 9

### 5-[1-(2,3-Dihydroindol-1-yl)-1-(3-methoxy-4-oxazol-5-yl-phenylamino)-methylene]-2,2-dimethyl[1,3]dioxane-4,6-dione

From Intermediate 1 (201 mg, 0.51 mmol), indoline (115 μl, 1.03 mmol) and mercury (II) chloride (139 mg, 0.51 mmol). The mixture was stirred at room temperature overnight. Purification by column chromatography on silica eluting with 3% methanol/dichloromethane yielded the title compound as a yellow solid (195 mg, 82%). TLC R_{f} 0.27 (3% MeOH/DCM). MS 404 [(M+H)-58]⁺.

¹H-NMR 300 MHz (d₆-DMSO) 10.4 (1H, s, br), 8.20 (2H, s), 7.4 (1H, m), 7.25 (1H, m), 7.10 (1H, m), 6.80-6.50 (4H, m), 4.00-3.88 (2H, m), 3.65-3.50 (3H, m), 3.90-3.00 (2H, m), 1.65-1.30 (6H, m).

### Intermediate 10

### 1-[(2,2-Dimethyl-4,6-dioxo[1,3]dioxan-5-ylidene)-(3-methoxy-4-oxazol-5-yl-phenylamino)methyl]piperidine-4-carboxylic acid methyl ester

From Intermediate 1 (500 mg, 1.28 mmol), methyl isonipecolate (185 mg, 1.28 mmol) and mercury (II) chloride (350 mg, 1.28 mmol). Purification by column chromatography on silica eluting with 10% methanol/dichloromethane yielded the title compound as an off-white solid (300 mg, 48%). TLC R_{f} 0.23 (10% MeOH/DCM). MS 428 [(M+H)-58]⁺.

### Intermediate 11

### 1-{[1-(2,2-Dimethyl-4,6-dioxo[1,3]dioxan-5-ylidene)-(3-methoxy-4-oxazol-5-yl-phenylamino)methyl]amino}butyric acid ethyl ester

From Intermediate 1 (500 mg, 1.28 mmol), ethyl 4-aminobutyrate hydrochloride (430 mg, 1.28 mmol), triethylamine (0.36 ml) and mercury (II) chloride (350 mg, 1.28 mmol). Purification by column chromatography on silica eluting with 75% ethyl acetate/heptane afforded the title compound as an off-white solid (620 mg, 100%). TLC R_{f} 0.74 (EtOAc).

### Intermediate 12

### 1-[(2,2-Dimethyl-4,6-dioxo[1,3]dioxan-5-ylidene)-(3-methoxy-4-oxazol-5-yl-phenylamino)methyl]piperidine-4-carboxylic acid amide

From Intermediate 1 (320 mg, 0.82 mmol), isonipecotamide (120 mg, 0.94 mmol) and mercury (II) chloride (260 mg, 0.94 mmol). Purification by column chromatography on silica eluting with 10% methanol/dichloromethane yielded the title compound as an off-white solid (225 mg, 58%). TLC R_{f} 0.17 (10% MeOH/DCM). MS 493 [M+Na]⁺.

### Intermediate 13

### 5-[(3-Methoxy-4-oxazol-5-yl-phenylamino)-(4-pyrrolidin-1-yl-piperidin-1-yl)methylene]-2,2-dimethyl[1,3]dioxane-4,6-dione

From Intermediate 1 (500 mg, 1.28 mmol), 4-(1-pyrrolidinyl)-piperidine (200 mg, 1.28 mmol) and mercury (II) chloride (350 mg, 1.28 mmol). Purification by column chromatography on silica eluting with 5% methanol/ethyl acetate afforded the title compound as an off-white solid (170 mg, 27%). TLC R_{f} 0.10 (10% MeOH/EtOAc).

### Intermediate 14

### 5-[(3.4-Dihydro-1H-isoquinolin-2-yl)-(3-methoxy-4-oxazol-5-yl-Phenylamino)methylene]-2,2-dimethyl[1,3]dioxane-4,6-dione

From Intermediate 1 (200 mg, 0.50 mmol), 1,2,3,4-tetrahydroisoquinoline (0.1 ml, 0.8 mmol) and mercury (II) chloride (140 mg, 0.50 mmol). The mixture was stirred at room temperature overnight. Purification by column chromatography on silica eluting with 2% methanol/dichloromethane yielded the title compound as a yellow solid (230 mg, 94%). HPLC RT 2.85 mins. MS 418 [(M+H)-58]⁺. ¹H-NMR 300 MHz (d₄-MeOH) 8.20 (1H, s), 7.75-7.70 (1H, d), 7.50 (1H, s), 7.28-7.15 (4H, m), 6.95-6.85 (2H, m), 4.85 (2H, m), 3.95-3.85 (5H, m), 3.18-3.07(2H, m),1.60-1.30 (6H, m).

### Intermediate 15

### 5-[(1,3-Dihydroisoindol-2-yl)-(3-methoxy-4-oxazol-5-yl-phenylamino)-methylene]-2,2-dimethyl[1,3]dioxane-4,6-dione

From Intermediate 1 (1 g, 2.56 mmol), isoindoline (0.44 ml, 3.85 mmol) and mercury (II) chloride (700 mg, 2.56 mmol). The mixture was stirred at room temperature overnight. Purification by column chromatography on silica eluting with 10% methanol/dichloromethane to afforded the title compound as a yellow solid (951 mg, 80%). MS 460 [M-H]⁻.

¹H-NMR 300 MHz (d₆-DMSO) 10.3 (1H, s, br), 8.50 (1H, s), 7.78-7.75 (1H, d), 7.59 (1H, s), 7.50-7.40 (4H, m), 7.00-7.90 (2H, m), 5.23-5.10 (4H, m), 3.95 (3H, s), 1.65-1.35 (6H, m).

### Intermediate 16

### 5-[(6,7-Dimethoxy-3,4-dihydro-1H-isoguinolin-2-yl)-(3-methoxy-4-oxazol-5-yl-phenylamino)methylene]-2,2-dimethyl[1,3]dioxane-4,6-dione

From Intermediate 1 (1 g, 2.56 mmol), 6,7-dimethoxy-1,2,3,4-tetrahydroisoquinoline.hydrogen chloride (0.88 g, 3.85 mmol), triethylamine (0.51 ml, 3.85 mmol) and mercury (II) chloride (700 mg, 2.56 mmol). The mixture was stirred at room temperature overnight. Purification by column chromatography on silica eluting with 5% methanol/dichloromethane yielded the title compound as a yellow solid (250 mg, 18%). HPLC RT 2.65 mins. MS 477 [(M+H)-58]⁺. ¹H-NMR 300 MHz (d₄-MeOH) 8.25 (1H, s), 7.77-7.72 (1H, d), 7.53 (1H, s), 7.97-6.75 (4H, m), 4.80-4.70 (2H, m), 3.95-3.90 (5H, m), 3.85 (3H, s), 3.80 (3H, s), 3.10-3.02 (2H, m), 1.65-1.30 (6H, m).

### Intermediate 17

### 5-[(5-Bromo-2,3-dihydroindol-1-yl)-(3-methoxy-4-oxazol-5-yl-phenylamino)methylene]-2,2-dimethyl[1,3]dioxane-4,6-dione

From Intermediate 1 (1 g, 2.56 mmol), 5-bromoindoline (762 mg, 3.85 mmol) and mercury (II) chloride (696 mg, 2.56 mmol). The mixture was stirred at room temperature overnight. Purification by column chromatography on silica eluting with 2% methanol/dichloromethane afforded the title compound as a yellow solid (703 mg, 51%). HPLC RT 3.47 mins. MS 482 [(M+H)-58]⁺.

### Intermediate 18

### 5-[(3-Methoxy-4-oxazol-5-yl-phenylamino)-(methylphenethylamino)-methylene]-2,2-dimethyl[1,3]dioxane-4,6-dione

From Intermediate 1 (500 mg, 1.28 mmol), N-methylphenylamine (0.37 ml, 2.56 mmol) and mercury (II) chloride (350 mg, 1.28 mmol). Purification by column chromatography on silica eluting with 5% methanol/dichloromethane afforded the title compound as an off-white solid (640 mg, 100%). TLC R_{f} 0.2 (EtOAc).

### Intermediate 19

### 5-[(Benzylmethylamino)-(3-methoxy-4-oxazol-5-yl-phenylamino)-methylene]-2,2-dimethyl[1,3]dioxane-4,6-dione

From Intermediate 1 (500 mg, 1.28 mmol), N-benzylmethylamine (0.33 ml, 2.56 mmol), and mercury (II) chloride (350 mg, 1.28 mmol). Purification by column chromatography on silica eluting with 10% methanol/dichloromethane afforded the title compound as an off-white solid (540 mg, 91%). TLC R_{f} 0.24 (10% MeOH/DCM).

### Intermediate 20

### 5-[(3-Methoxy-4-oxazol-5-yl-phenylamino)-(4-phenylpiperidin-1-yl)-methylene]-2,2-dimethyl[1,3]dioxane-4,6-dione

From Intermediate 1 (1.01 g, 2.59 mmol), 4-phenyl piperidine (0.417 g, 2.59 mmol), and mercury (II) chloride (703 mg, 2.59 mmol). Purification by column chromatography on silica eluting with 10% methanol/dichloromethane yielded the title compound as a tan solid (693 mg, 53%). HPLC RT 3.21 mins. MS 504 [M+H]⁺.

### Intermediate 21

### 5-[(3-Methoxy-4-oxazol-5-yl-phenylamino)-(2-methyl-2,3-dihydroindol-1-yl)methylene]-2,2-dimethyl[1,3]dioxane-4,6-dione

From Intermediate 1 (1 g, 2.56 mmol), 2-methylindoline (0.5 ml, 3.85 mmol), and mercury (II) chloride (700 mg, 2.56 mmol). The reaction mixture was stirred at room temperature overnight. Purification by column chromatography on silica eluting with 5% methanol/dichloromethane yielded a brown solid (1.28 g). This was recrystalised from 5% methanol/dichloromethane to give the title compound as a beige solid (1 g, 82%). HPLC RT 3.30 mins. MS 418 [(M+H)-58]⁺.

### Intermediate 22

### 5-[1-(3-Methoxy-4-oxazol-5-yl-phenylamino)-1-morpholin-4-yl-methylene]-2,2-dimethyl[1,3]dioxane-4,6-dione

A mixture of Intermediate 1 (149 mg, 0.38 mmol), morpholine (5 ml) and mercury (II) chloride (104 mg, 0.38 mmol) were stirred at room temperature for 18 hrs. The solvent was removed in vacuo and the residue purified by column chromatography on silica eluting 0-5% methanol/dichloromethane to yield the title compound as a yellow solid (31 mg, 19%). TLC R_{f} 0.32 (5% MeOH/DCM). MS 430 [M+H]⁺. ¹H-NMR 300 MHz (d₆-DMSO) 10.0 (1H, s, br), 8.25 (1H, s), 7.48 (1H, d), 7.30 (1H, s), 6.65-6.60 (2H, m), 3.73 (3H, m), 3.60 (4H, m), 3.40-3.30 (4H, m), 3.78 (3H, s), 1.25-1.35 (6H, s).

### Intermediate 23

### 5-{(3-Methoxy-4-oxazol-5-yl-phenylamino)[tetrahydropyran-2-ylmethyl)-amino]methylene}-2,2-dimethyly[1,3]dioxane-4,6-dione

A mixture of Intermediate 1 (376 mg, 0.965 mmol) and 2-aminomethyltetrahydropyran (11 mg, 0.965 mmol) in tetrahydrofuran (10 ml) were stirred at room temperature for 18 hrs. The solvent was removed in vacuo to yield the title compound as an off-white solid (440 mg, 100%). HPLC RT 3.63 mins. MS 400 [(M+H)-58]⁺.

Intermediates 24-40 were prepared in a similar manner to Intermediate 23:

### Intermediate 24

### 5-{(3-Methoxy-4-oxazol-5-yl-phenylamino)[tetrahydrofuran-2-ylmethyl)-amino]methylene}-2,2-dimethyl[1,3]dioxane-4,6-dione

From Intermediate 1 (334 mg, 0.856 mmol), and tetrahydrofurfurylamine (0.088 ml, 0.856 mmol). The solvent was removed in vacuo to yield the title compound as a yellow solid (380 mg, 100%). HPLC RT 3.34 mins. MS 444 [M+H]⁺.

### Intermediate 25

### 5-[1-((S)-2-Methoxy-1-methylethylamino)-1-(3-methoxy-4-oxazol-5-yl-phenylamino)methylene]-2,2-dimethyl[1,3]dioxane-4,6-dione

From Intermediate 1 (500 mg, 1.28 mmol), and (S)-(+)-1-methoxy-propylamine (230 mg, 2.56 mmol). The solvent was removed in vacuo to yield the title compound as a yellow solid (540 mg, 98%). TLC R_{f} 0.54 (75% EtOAc/Hexane).

### Intermediate 26

### 5-[1-(3-Methoxy-4-oxazol-5-yl-phenylamino)-1-(2-methylpyrrolidin-1-yl)-methylene]-2,2-dimethyl[1,3]dioxane-4,6-dione

From Intermediate 1 (500 mg, 1.28 mmol), and 2-methylpyrrolidine (0.26 ml, 2.56 mmol). The solvent was removed in vacuo to yield the title compound as a yellow solid (540 mg, 100%). TLC R_{f} 0.43 (10% MeOH/DCM).

### Intermediate 27

### 5-[(3-Methoxy-4-oxazol-5-yl-phenylamino)(3-nitrobenzylamino)-methylene]-2,2-dimethyl[1,3]dioxane-4,6-dione

From Intermediate 1 (1.03 g, 2.64 mmol), 3-nitrobenzylamine hydrochloride (498 mg, 2.64 mmol) and triethylamine (0.37 ml, 2.64 mmol). The reaction mixture was washed with water (2×20 ml), dried over magnesium sulphate, filtered and the solvent removed in vacuo to yield the title compound as a yellow gum (1.3 g, 100%). HPLC RT 3.58 mins. MS 495 [M+H]⁺.

### Intermediate 28

### 4-({[(2,2-Dimethyl-4,6-dioxo[1,3]dioxan-5-ylidene)-(3-methoxy-4-oxazol-5-yl-phenylamino)methyl]amino)methyl]amino}methyl)benzonitrile

From Intermediate 1 (1.03 g, 2.64 mmol), 4-cyanobenzylamine hydrochloride (446 mg, 2.64 mmol) and triethylamine (0.37 ml, 2.64 mmol). The reaction mixture was washed with water (2×20 ml), dried over magnesium sulphate, filtered and the solvent removed in vacuo to yield the title compound as a yellow gum (1.3 g, 100%). HPLC RT 3.49 mins. MS 475 [M+H]⁺.

### Intermediate 29

### 5-[(2-Imidazol-1-yl-ethylamino)-(3-methoxy-4-oxazol-5-yl-phenylamino)-methylene]-2,2-dimethyl[1,3]dioxane-4,6-dione

From Intermediate 1 (500 mg, 1.28 mmol), N-aminoethylimidazole (350 mg, 1.92 mmol) and triethylamine (0.54 ml, 3.84 mmol). The reaction mixture was heated at reflux overnight. The solvent was removed in vacuo and the residue purified by column chromatography on silica eluting with 5% methanol/dichloromethane to yield the title compound as an off white solid (370 mg, 64%). TLC R_{f} 0.40 (10% MeOH/DCM).

### Intermediate 30

### 3-({[(2,2-Dimethyl-4,6-dioxo[1,3]dioxan-5-ylidene)-(3-methoxy-4-oxazol-5-yl-phenylamino)methyl]amino)methyl]amino}methyl)benzoic acid methyl ester

From Intermediate 1 (723 mg, 1.85 mmol) and 3-aminomethyl benzoic acid methyl ester (306 mg, 1.85 mmol). The solvent was removed in vacuo to yield the title compound as a yellow foam (985 mg, 100%). HPLC RT 3.70 mins. MS 506 [M+H]⁺.

### Intermediate 31

### 5-[(2-Methoxybenzylamino)-(3-methoxy-4-oxazol-5-yl-phenylamino)-methylene1-2,2-dimethyl[1,3]dioxane-4,6-dione

From Intermediate 1 (1 g, 2.56 mmol) and 2-methoxybenzylamine (0.33 ml, 2.56 mmol). The solvent was removed in vacuo to yield the title compound as a yellow foam (4.2 g, 100%). HPLC RT 3.92 mins. MS 422 [(M+H)-58]⁺.

### Intermediate 32

### 5-[(2-Bromobenzylamino)(3-methoxy-4-oxazol-5-yl-phenylamino)-methylene]-2,2-dimethyl1,3]dioxane-4,6-dione

From Intermediate 1 (1 g, 2.56 mmol) and 2-bromobenzylamine hydrochloride (0.57 g, 2.56 mmol). The reaction was stirred at room temperature for 80 hours. The solvent was removed in vacuo and the residue partitioned between dichloromethane (60 ml) and water (20 ml). The organic layer was dried over magnesium sulphate, filtered and the filtrate evaporated in vacuo to yield the title compound as an off-white solid (1.51 g, 100%). HPLC RT 4.03 mins. MS 529 [M+H]⁺.

### Intermediate 33

### 5-[[Benzo[1,3]dioxol-5-ylmethyl)amino(3-methoxy-4-oxazol-5-yl-phenylamino)methylene]-2,2-dimethyl[1,3]dioxane-4,6-dione

From Intermediate 1 (500 mg, 1.28 mmol) and 3,4-(methylene-dioxy)-benzylamine (0.32 ml, 2.56 mmol). The reaction mixture was heated at reflux overnight. The solvent was removed in vacuo and the residue purified by column chromatography on silica eluting with 50-75% ethyl acetate/heptane to yield the title compound as a yellow solid (600 mg, 95%). TLC R_{f} 0.70 (5% MeOH/DCM).

### Intermediate 34

### 5-{(3-Methoxy-4-oxazol-5-yl-phenylamino)[(naphthalene-1-ylmethyl)-amino]methylene}-2,2-dimethyl[1,3]dioxane-4,6-dione

From Intermediate 1 (500 mg, 1.28 mmol) and 1-naphthlene-methylamine (0.38 ml, 2.56 mmol). The reaction was heated at reflux overnight. The solvent was removed in vacuo and the residue purified by column chromatography on silica eluting with 50-75% ethyl acetate/heptane to yield the title compound as a yellow oil (610 mg, 95%). TLC R_{f} 0.38 (50% EtOAc/Hexane).

### Intermediate 35

### 5-[(2,4-Dichlorobenzylamino)-3-methoxy-4-oxazol-5-yl-phenylamino)-methylene]-2,2-dimethyl[1,3]dioxane-4,6-dione

From Intermediate 1 (1.05 g, 2.7 mmol) and 2,4-dichloro-benzylamine (0.32 ml, 2.56 mmol). The reaction was stirred at room temperature for 18 hours. The solvent was removed in vacuo to yield the title compound as a yellow solid (1.4 g, 100%). HPLC RT 4.39 mins. MS 520 [M+H]⁺.

### Intermediate 36

### 5-[3-Methoxy-4-oxazol-5-yl-phenylamino)(4-phenoxybenzylamino)-methylene]-2,2-dimethyl[1,3]dioxane-4,6-dione

From Intermediate 1 (500 mg, 1.28 mmol) and 4-phenoxybenzylamine (0.51 g, 2.56 mmol). The reaction was heated at reflux overnight. The solvent was removed in vacuo and the residue purified by column chromatography on silica eluting with 75% ethyl acetate/heptane to yield the title compound as a yellow solid (630 mg, 91%). TLC R_{f} 0.43 (50% EtOAc/Hexane).

### Intermediate 37

### 5-[[(Biphenyl-3-ylmethyl)amino](3-methoxy-4-oxazol-5-yl-phenylamino)-(4-phenoxybenzylamino)methylene]-2,2-dimethyl[1,3]dioxane-4,6-dione

From Intermediate 1 (900 mg, 2.3 mmol), and 3-phenylbenzylamine (0.5 g, 2.7 mmol). Purification by column chromatography on silica eluting with 50% ethyl acetate/heptane afforded the title compound as an pale yellow solid (1.2 g, 100%). TLC R_{f} 0.26 (50% EtOAc/Heptane). MS 526 [M+H]⁺.

### Intermediate 38

### [3-([[(2,2-Dimethyl-4,6-dioxo[1,3]dioxan-5-ylidene)-(3-methoxy-4-oxazol-5-yl-phenylamino)methyl]amino]methyl)benzyl]carbamic acid benzyl ester

From Intermediate 1 (1.5 g, 3.7 mmol) and (3-aminomethyl-benzyl)-carbamic acid benzyl ester (1.0 g, 3.7 mmol). Purification by column chromatography on silica eluting with 50-100% ethyl acetate/heptane afforded the title compound as a pale yellow solid (825 mg, 35%). TLC R_{f} 0.13 (50% EtOAc/Heptane). MS 635 [M+Na]⁺.

### Intermediate 39

### 5-[[(Benzofuran-2-yl methyl)amino]-3-methoxy-4-oxazol-5-yl-phenylamino)methylene]-2,2-dimethyl[1,3]dioxane-4,6-dione

From Intermediate 1 (780 mg, 2 mmol) and 2-aminomethyl-benzofuran (CAS 37798-05-3) (645 mg, 2 mmol). Purification by column chromatography on silica eluting with 50% ethyl acetate/heptane afforded the title compound as a yellow solid (734 mg, 75%). TLC R_{f} 0.3 (50% EtOAc/Heptane). MS 490 [M+H]⁺.

### Intermediate 40

### 5-[[(Cyclohexylmethyl)amino]-3-methoxy-4-oxazol-5-yl-phenylamino)-methylene]-2,2-dimethyl[1,3]dioxane-4,6-dione

From Intermediate 1 (500 mg, 1.28 mmol) and cyclohexanemethylamine (0.33 ml, 2.56 mmol). Purification by column chromatography on silica eluting with 20-40% ethyl acetate/heptane afforded the title compound as a pale yellow solid (490 mg, 84%). TLC R_{f} 0.58 (50% EtOAc/Heptane).

### Intermediate 41

### 2-Amino-7-methoxy-6-oxazol-5-yl-1H-quinolin-4-one

Intermediate 4 (225 mg, 0.63 mmol) was diluted with diphenyl ether (6 ml) and heated to reflux for 10 minutes. The reaction mixture was diluted with hexane and the precipitate formed collected by filtration. The solid was dissolved in methanol and filtered, the solvent removed in vacuo and the resulting solid dried in a vacuum oven overnight to yield the title compound (130 mg, 81%). TLC R_{f} 0.11 (10% MeOH/DCM). MS 258 [M+H]⁺.

¹H-NMR 300 MHz (d₄-MeOH) 8.64 (1H, s), 8.40 (1H, s), 7.62 (1H, s), 7.06 (1H, s), 5.72 (1H, s), 4.20 (2H, s), 3.42 (3H, m).

### EXAMPLE 1

### 7-Methoxy-6-oxazol-5-yl-2-phenylamino-1H-quinolin-4-one

A mixture of Intermediate 2 (408 mg, 0.94 mmol) in diphenylether (5 ml) was stirred and heated at reflux for 30 minutes. The reaction was allowed to cool to room temperature and the resultant solid was purified by column chromatography on silica eluting with 10% methanol/dichloromethane to yield the title compound (71 mg, 24%). TLC R_{f} 0.29 (10% MeOH/DCM). MS 334 [M+H]⁺. ¹H-NMR 400 MHz (d₄-MeOH) 8.50 (1H, s), 8.28 (1H, s), 7.55 (1H, s) 7.45 (2H, m), 7.30 (2H, m), 7.25 (1H, m), 7.10 (1H, s), 5.82 (1H, s), 4.95-4.75 (1H, s, br), 4.70-4.55 (1H, s, br), 4.05 (3H, s).

The compounds of Examples 2-15 were prepared in a similar manner to the compound of Example 1:

### EXAMPLE 2

### 7-Methoxy-6-oxazol-5-yl-2-(pyridin-3-ylamino)-1H-quinolin-4-one

From Intermediate 3 (48 mg, 0.01 mmol). Purification by column chromatography on silica eluting with 5% methanol/dichloromethane yielded the title compound (2 mg, 5%). TLC R_{f} 0.29 (5% MeOH/DCM). MS 335 [M+H]⁺. ¹H-NMR 400 MHz (d₄-MeOH) 8.50 (1H, m), 8.40 (1H, s), 8.20 (1H, m) 8.15 (1H, s), 7.78-7.88 (1H, m), 7.43 (1H, s), 7.35 (1H, m), 7.02 (1H, s), 5.80 (1H, s, br), 3.98 (3H, s).

### EXAMPLE 3

### 7-Methoxy-2-morpholin-4-yl-6-oxazol-5-yl-1H-quinolin-4-one

From Intermediate 22 (143 mg, 0.33 mmol). Purification by column chromatography on silica eluting with 0-10% methanol/dichloromethane yielded the title compound as an off-white solid (13 mg, 12%). TLC R_{f} 0.09 (5% MeOH/DCM). MS 328 [M+H]⁺. ¹H-NMR 400 MHz (d₆-DMSO) mixture of 1H and 4H tautomers 11.15 (1H, s, br), 10.73 (1H, s, br), 8.42 (2H, s) 8.25-8.18 (2H, d), 7.53 (2H, s), 7.15 (1H, s), 7.02 (1H, s), 6.30 (1H, s), 5.40 (1H, s), 4.0 (6H, s), 3.80-3.55 (16H, m).

### EXAMPLE 4

### 7-Methoxy-6-oxazol-5-yl-2-[(pyridin-3-ylmethyl)amino]1H-quinolin-4-one

From Intermediate 6 (255 mg, 0.57 mmol). Purification by column chromatography on silica eluting with 20% methanol/dichloromethane afforded the title compound as an off-white solid (10 mg, 5.3%). MS 349 [M+H]⁺. ¹H-NMR 300 MHz (d₄-MeOH) 8.60 (1H, s), 8.45 (1H, d), 8.40 (1H, s), 8.25 (1H, s), 7.90 (1H, d), 7.50 (1H, s), 7.40-7.50 (1H, m), 6.95 (1H, s), 5.50 (1H, s), 4.65 (2H, s), 4.05 (3H, s).

### EXAMPLE 5

### 7-Methoxy-6-oxazol-5-yl-2-[(furan-2-ylmethyl)amino]1H-quinolin-4-one

From Intermediate 7 (250 mg, 0.57 mmol). Purification by column chromatography on silica eluting with 10% methanol/dichloromethane afforded the title compound as an off-white solid (17 mg, 8.7%). MS 338 [M+H]⁺. ¹H-NMR 300 MHz (d₄-MeOH) 8.60 (1H, s), 8.35 (1H, s), 7.50 (1H, s), 7.45 (1H, s), 7.10 (1H, s), 6.45 (2H, s), 5.80(1H, s), 4.60 (2H, s), 4.25 (3H, s). EXAMPLE 6

### 4-(7-Methoxy-6-oxazol-5-yl-4-oxo-1,4-dihydro-quinolin-2-yl)piperazine-1-carboxylic acid tert-butyl ester

From Intermediate 5 (100 mg, 0.189 mmol). The reaction mixture was diluted with hexane and the precipitate collected by filtration. Purification by column chromatography on silica eluting with 10% methanol/dichloromethane afforded the title compound (120 mg, 33%). TLC R_{f} 0.21 (10% MeOH/DCM). MS 427 [M+H]⁺.

¹H-NMR 300 MHz (d₄-MeOH) 8.56 (1H, s), 8.33 (1H, s), 8.31 (1H, s), 7.61 (1H, s), 7.23 (1H, s), 5.82 (1H, s), 4.15 (3H, s), 3.72-3.65 (4H, m) 3.60-3.52 (4H, m), 1.58 (9H, s).

### EXAMPLE 7

### 2-[(1-Ethylpyrrolidin-2-ylmethyl)amino]-7-methoxy-6-oxazol-5-yl-1H-quinolin-4-one; formate salt

From Intermediate 8 (290 mg, 0.62 mmol). The reaction mixture was diluted with hexane, and the precipitate collected by filtration. Purification by preparative HPLC (Method A) afforded the title compound as an off-white solid (6.4 mg, 3%). HPLC RT 1.24 mins. MS 369 [M+H]⁺.

### EXAMPLE 8

### 2-(2,3-Dihydroindol-1-yl)-7-methoxy-6-oxazol-5-yl-1H-quinolin-4-one

From Intermediate 9 (187 mg, 0.41 mmol). Purification by column chromatography on silica eluting with 5-10% methanol/dichloromethane yielded the title compound as a pale green solid (83 mg, 57%). TLC R_{f} 0.11 (5% MeOH/DCM). MS 360 [M+H]⁺. ¹H-NMR 400 MHz (d₆-DMSO) 11.50 (1H, s), 8.86-8.82 (1H, d), 8.63 (1H, s) 8.43 (1H, s), 7.72 (1H, s), 7.44-7.32 (3H, m), 7.10-7.00 (1H, m), 6.56 (1H, s), 4.30-4.18 (5H, m), 3.40-3.31 (2H, m).

### EXAMPLE 9

### 1-(7-Methoxy-6-oxazol-5-yl-4-oxo-1,4-dihydro-quinolin-2-yl)piperidine-4-carboxylic acid methyl ester

From Intermediate 10 (300 mg, 0.61 mmol). The reaction mixture was heated at 200° C. for 2 hours. Purification by column chromatography on silica eluting with 0-10% methanol/dichloromethane afforded the title compound as an off-white solid (185 mg, 78%). TLC R_{f} 0.25 (10% MeOH/DCM). HPLC RT 1.91 mins. MS 384 [M+H]⁺.

### EXAMPLE 10

### 7-Methoxy-6-oxazol-5-yl-2-[(tetrahydropyran-2-ylmethyl)amino]-1H-quinolin-4-one; formate salt

From Intermediate 23 (441 mg, 0.965 mmol). The reaction mixture was heated at 220°0 C. for 4 hours. Purification by preparative HPLC (method A) afforded the title compound as an off-white solid (36 mg, 11%). HPLC RT 1.95 mins. MS 356 [M+H]⁺.

### EXAMPLE 11

### 7-Methoxy-6-oxazol-5-yl-2-[(tetrahydrofuran-2-ylmethyl)amino]-1H-quinolin-4-one

From Intermediate 24 (379 mg, 0.856 mmol). The reaction mixture was heated at 220° C. for 4 hours. Purification by preparative HPLC (method A) afforded the title compound as an off-white solid (38 mg, 13%). HPLC RT 1.75 mins. MS 342 [M+H]⁺.

### EXAMPLE 12

### 7-Methoxy-6-oxazol-5-yl-2-(2-oxopyrrolidin-1-yl)-1H-quinolin-4-one

From Intermediate 11 (600 mg, 0.61 mmol). The reaction mixture was heated at 190° C. for 90 minutes. Purification by column chromatography on silica eluting with 0-10% methanol/dichloromethane followed by trituration with dichloromethane afforded the title compound as an off-white solid (37 mg, 9%). TLC R_{f} 0.38 (10% MeOH/DCM). HPLC RT 2.04 mins. MS 326 [M+H]⁺.

### EXAMPLE 13

### 7-Methoxy-2-((S)-2-methoxy-1-methylethylamino)-6-oxazol-5-yl-1H-quinolin-4-one

From Intermediate 25 (600 mg, 0.61 mmol). The reaction mixture was heated at 200° C. for 6 hours. Purification by column chromatography on silica eluting with 0-20% methanol/dichloromethane followed by trituration with diethyl ether afforded the title compound as an off white solid (42 mg, 11%). TLC R_{f} 0.09 (10% MeOH/DCM). HPLC RT 1.79 mins. MS 330 [M+H]⁺.

### EXAMPLE 14

### 7-Methoxy-2-(2-methylpyrrolidin-1-yl)-6-oxazol-5-yl-1H-quinolin-4-one

From Intermediate 26 (540 mg, 1.26 mmol). The reaction mixture was heated at 200° C. for one hour. Purification by column chromatography on silica eluting with 0-20% methanol/dichloromethane followed by trituration with dichloromethane/diethyl ether afforded the title compound as an off-white solid (190 mg, 46%). TLC R_{f} 0.26 (10% MeOH/DCM). HPLC RT 1.85 mins. MS 326 [M+H]⁺.

### EXAMPLE 15

### 1-(7-Methoxy-6-oxazol-5-yl-4-oxo-1 4-dihydroquinolin-2-yl)piperidine-4-carboxylic acid amide

From Intermediate 12 (900 mg, 1.91 mmol). The reaction mixture was heated at 200° C. for one hour. The reaction was diluted with heptane (100 ml) and the solid obtained by filtration. Purification by column chromatography on silica eluting with 0-20% methanol/dichloromethane afforded the title compound as an off-white solid (105 mg, 15%). TLC R_{f} 0.17 (10% MeOH/DCM). HPLC RT 1.59 mins. MS 369 [M+H]⁺.

### EXAMPLE 16

### 7-Methoxyoxazol-5-yl-2-(4-pyrrolidin-1-yl-piperidin-1-yl)-1H-quinolin-4-one

A solution of Intermediate 13 (170 mg, 0.34 mmol) in diphenyl ether/N-methylpyrrolidinone (20 ml/2 ml) was heated at 250° C. in a microwave reactor for 20 mins. The reaction was diluted with heptane and the solid filtered off. The solid was recrystallised from methanol/dichloromethane/heptane to afford the title compound as an off-white solid (75 mg, 56%). TLC R_{f} 0.05 (20% MeOH/DCM). HPLC RT 1.26mins. MS 395 [M+H]⁺. The compounds of Examples 17-38 were prepared in a similar manner to the compound of Example 16:

### EXAMPLE 17

### 7-Methoxy-2-(3-nitrobenzylamino)-6-oxazol-5-yl-1H-quinolin-4-one

From Intermediate 27 (1.3 g, 2.64 mmol). Purification by column chromatography on silica eluting with 0-20% methanol/dichloromethane afforded the title compound as a brown solid (539 mg, 52%). TLC R_{f} 0.15 (10% MeOH/DCM). MS 392 [M+H]⁺.

### EXAMPLE 18

### 2-(Dihydro-1H-isoguinolin-2-yl)-7-methoxy-6-oxazol-5-yl-1H-quinolin-4-one

From Intermediate 14 (147 mg, 0.31 mmol). The mixture was diluted with hexane (100 ml) and filtered. The resulting solid was purified by column chromatography on silica eluting with 8% methanol/dichloromethane to afford the title compound as a brown solid (26 mg, 23%). HPLC RT 2.17 mins. MS 374 [M+H]⁺. ¹H-NMR 400 MHz (d₄-MeOH) 8.50 (1H, s) 8.25 (1H, s), 7.54 (1H, s), 7.29-7.22 (5H, m), 5.81 (1H, s), 4.66 (2H, s), 4.10 (3H, s), 3.80-3.73 (2H, m), 3.10-3.03 (2H, m).

### EXAMPLE 19

### 4-[(7-Methoxy-6-oxazol-5-yl-4-oxo-1,4-dihydroquinolin-2-ylamino-methyl]benzonitrile

From Intermediate 28 (1.2 g, 2.65 mmol). Purification by column chromatography on silica eluting with 0-20% methanol/dichloromethane afforded the title compound as a light brown solid (502 mg, 51%). TLC R_{f} 0.18 (10% MeOH/DCM). MS 373 [M+H]⁺.

### EXAMPLE 20

### 2-(2-Imidazol-1-yl-ethylamino)-7-methoxy-6-oxazol-5-yl-1H-quinolin-4-one; formate salt

From Intermediate 29 (0.35 g, 0.77 mmol). The mixture was diluted with hexane and the resulting brown solid filtered off. Purification by preparative HPLC (method A) afforded the title compound as pale brown solid (90 mg, 33%). HPLC RT 1.21 mins. MS 352 [M+H]⁺.

### EXAMPLE 21

### 4-[7-Methoxy-6-oxazol-5-yl-4-oxo-1,4-dihydro-quinolin-2-ylamino)-methyl]benzoic acid methyl ester

From Intermediate 30 (985 mg, 1.94 mmol). Purification by column chromatography on silica eluting with 0-20% methanol/dichloromethane afforded the title compound as a tan solid (269 mg, 34%). HPLC RT 2.08 mins. MS 406 [M+H]⁺.

### EXAMPLE 22

### 7-Methoxy-2-(2-methoxybenzylamino)-6-oxazol-5-yl-1H-quinolin-4-one

From Intermediate 31 (1.42 g, 3.0 mmol). Purification by column chromatography on silica eluting with 10-20% methanol/dichloromethane followed trituration in methanol/dichloromethane/heptane and washing with methanol afforded the title compound as a yellow solid (39 mg, 12%). TLC R_{f} 0.40 (10% MeOH/DCM). MS 378 [M+H]⁺.

### EXAMPLE 23

### 2-(1,3-Dihydroisoindol-2-yl)-7-methoxy-6-oxazol-5-yl-1H-quinolin-4-one

From Intermediate 15 (950 mg, 2.06 mmol). Purification by column chromatography on silica eluting with 10-20% methanol/dichloromethane yielded a brown solid. This was recrystallised from 5% methanol/dichloromethane to give the title compound as a beige solid (38 mg, 5%). HPLC RT 2.07 mins. MS 360 [M+H]⁺. ¹H-NMR 400 MHz (d₄-MeOH) 8.45 (1H, s) 8.00 (1H, s), 7.43 (1H, s), 7.34 (4H, s), 7.17 (1H, s), 5.53 (1H, s), 4.80-4.70 (4H, s), 4.10 (3H, s).

### EXAMPLE 24

### 2-(2-Bromobenzylamino)-7-methoxy-6-oxazol-5-yl-1H-quinolin-4-one

From Intermediate 32 (990 mg, 1.88 mmol). Purification by column chromatography on silica eluting with 0-10% methanol/dichloromethane afforded the title compound as an off-white solid (216 mg, 27%). HPLC RT 2.18 mins. MS 427 [M+H]⁺.

### EXAMPLE 25

### 2-[(Benzo[1,3]dioxol-5-ylmethyl)amino]-7-methoxy-6-oxazol-5-yl-1H-quinolin-4-one; formate salt

From Intermediate 33 (0.6 g, 1.22 mmol). Purification by column chromatography on silica eluting with 0-20% methanol/dichloromethane gave a green-brown solid. Preparative HPLC (method A) afforded the title compound as an off-white solid (44 mg, 9%). TLC R_{f} 0.13 (10% MeOH/DCM). MS 392 [M+H]⁺.

### EXAMPLE 26

### 2-(6,7-Dimethoxy-3,4-dihydro-1H-isoguinolin-2-yl)-7-methoxy-6-oxazol-5-yl-1H-quinolin-4-one

From Intermediate 16 (250 mg, 0.47 mmol). Purification by column chromatography on silica eluting with 5-10% methanol/dichloromethane yielded a brown solid. This was recrystalised from 5% methanol/dichloromethane to give the title compound as a beige solid (54 mg, 27%). HPLC RT 2.10 mins. MS 434 [M+H]⁺. ¹H-NMR 400 MHz (d₄-MeOH) 8.45 (1H, s) 8.27 (1H, s), 7.53 (1H, s), 7.22 (1H, s), 6.82 (2H, m), 4.53 (2H, s), 4.08 (3H, s), 3.81 (6H, s), 3.72-3.67 (2H, m), 3.00-2.93 (2H, m).

### EXAMPLE 27

### 2-(5-Bromo-2,3-dihydroindol-1-yl)-7-methoxy-6-oxazol-5-yl-1H-quinolin-4-one

From Intermediate 17 (990 mg, 2.1 mmol). Purification by column chromatography on silica eluting with 5% methanol/dichloromethane gave the title compound as a brown solid (128 mg, 23%). HPLC RT 2.46 mins. MS 374 [M+H]⁺. ¹H-NMR 400 MHz (d₄-MeOH) 8.52 (1H, s) 8.28 (1H, s), 7.55 (1H s), 7.34-7.20 (4H, m), 7.04-6.96 (1H, m), 6.15 (1H, s), 4.71-4.63 (1H, m), 4.11 (3H, s), 3.54-3.41 (1H, dd), 2.85-2.78 (1H, dd), 1.45-1.35 (3H, d).

### EXAMPLE 28

### 7-Methoxy-2-[(napthalen-1-ylmethyl)amino]-6-oxazol-5-yl-1H-quinolin-4-one

From Intermediate 34 (600 mg, 1.2 mmol). Hexane was added to the reaction mixture and the resulting solid filtered off. Purification by column chromatography on silica followed by trituration in diethyl ether afforded the title compound as a beige solid (95 mg, 20%). TLC R_{f} 0.29 (10% MeOH/DCM). MS 398 [M+H]⁺.

### EXAMPLE 29

### 2-(2,4-Dichlorobenzylamino)-7-methoxy-6-oxazol-5-yl-1H-quinolin-4-one

From Intermediate 35 (1.4 g, 2.7 mmol). Purification by column chromatography on silica eluting with 0-20% methanol/dichloromethane afforded the title compound as a light brown solid (459 mg, 41%). TLC R_{f} 0.20 (10% MeOH/DCM). MS 416 [M+H]⁺.

### EXAMPLE 30

### 7-Methoxy-6-oxazol-5-yl-2-(4-phenoxybenzylamino)-1H-quinolin-4-one

From Intermediate 36 (600 mg, 1.11 mmol). Purification by column chromatography on silica eluting with 0-15% methanol/dichloromethane followed by trituration in methanol/diethyl ether afforded the title compound as a beige solid (115 mg, 24%). TLC R_{f} 0.27 (10% MeOH/DCM). MS 440 [M+H]⁺.

### EXAMPLE 31

### 7-Methoxy-2-(methylphenethylamino)-6-oxazol-5-yl-1H-quinolin-4-one

From Intermediate 31 (640 mg, 1.34 mmol). Purification by column chromatography on silica eluting with 0-20% methanol/dichloromethane followed by trituration in diethyl ether afforded the title compound as a beige solid (290 mg, 57%). TLC R_{f} 0.29 (10% MeOH/DCM). MS 376 [M+H]⁺.

### EXAMPLE 32

### 2-[(Biphenyl-3-ylmethyl)amino]-7-methoxy-6-oxazol-5-yl-1H-quinolin-4-one

From Intermediate 37 (1.2 g, 2.3 mmol). Purification by column chromatography on silica eluting with 2-10% methanol/dichloromethane followed by preparative HPLC (method A) afforded the title compound as an off-white solid (214 mg, 22%). TLC R_{f} 0.35 (10% MeOH/DCM). MS 424 [M+H]⁺.

### EXAMPLE 33

### {3-[(7-Methoxy-6-oxazol-5-yl-4-oxo-1.4-dihydro-quinolin-2-ylamino)-methyl]benzyl}carbamic acid benzyl ester

From Intermediate 38 (800 mg, 1.3 mmol). Purification by column chromatography on silica eluting with 5-10% methanol/dichloromethane followed by prep HPLC (method A) afforded the title compound as an off white solid (85 mg, 13%). TLC R_{f} 0.28 (10% MeOH/DCM). MS 511 [M+H]⁺.

### EXAMPLE 34

### 2-(Benzylmethylamino)-7-methoxy-6-oxazol-5-yl-1H-quinolin-4-one

From Intermediate 19 (540 mg, 1.17 mmol). Purification by column chromatography on silica eluting with 0-15% methanol/dichloromethane followed by trituration in diethyl ether afforded the title compound as a salmon pink solid (270 mg, 64%). TLC R_{f} 0.50 (20% MeOH/DCM). MS 362 [M+H]⁺.

### EXAMPLE 35

### 7-Methoxy-6-oxazol-5-yl-2-(4-phenyl piperidin-1-yl)-1H-quinolin-4-one

From Intermediate 20 (693 mg, 1.38 mmol). Purification by column chromatography on silica eluting with 0-10% methanol/dichloromethane afforded the title compound as a brown solid (154 mg, 28%). HPLC RT 2.42mins. MS 402 [M+H]⁺.

### EXAMPLE 36

### 2-[(Benzofuran-2-ylmethyl)amino-7-methoxy-6-oxazol-5-yl-quinolin-4-one

From Intermediate 39 (734 mg, 1.5 mmol). Purification by column chromatography on silica eluting with 0-20% methanol/dichloromethane afforded the title compound as a brown solid (228 mg, 39%). TLC R_{f} 0.20 (10% MeOH/DCM). MS 388 [M+H]⁺.

### EXAMPLE 37

### 7-Methoxy-2-(2-methyl-2,3-dihydroindol-1-yl)-6-oxazol-5-yl-1H-quinolin-4-one

From Intermediate 21 (700 mg, 1.3 mmol). Purification by column chromatography on silica eluting with 10% methanol/dichloromethane afforded the title compound as a brown solid (128 mg, 23%). HPLC RT 2.91 mins. MS 438 [M+H]⁺. ¹H-NMR 400 MHz (d₄-MeOH) 8.50 (1H, s) 8.19 (1H, s), 7.74 (1H, s), 7.51 (1H, s), 7.36-7.30 (2H, m), 7.16 (1H, s), 6.10 (1H, s), 4.18-4.08 (5H, m), 3.22-3.15 (2H, m).

### EXAMPLE 38

### 2-(Cyclohexylmethylamino)-7-methoxy-6-oxazol-5-yl-1H-quinolin-4-one

From Intermediate 40 (490 mg, 1.07 mmol). Purification by column chromatography eluting with 0-20% methanol/dichloromethane followed by trituration in dichloromethane/diethyl ether afforded the title compound as a pink solid (130 mg, 34%). TLC R_{f} 0.28 (10% MeOH/DCM). MS 354 [M+H]⁺.

### EXAMPLE 39

### 7-Methoxy-6-oxazol-5-yl-2-piperazin-1-yl-1H-quinolin-4-one; dihydrochloride

The compound of Example 6 (90 mg, 2.11 mmol) was dissolved in methanol (20 ml) with stirring. 1.0M Hydrochloric acid (10 ml) was added and the reaction stirred at room temperature for 2 hours. Mixture was concentrated in vacuo to give the title compound (91 mg). MS 327 [M+H]⁺. ¹H-NMR 300 MHz (d₆-DMSO) 8.68 (1H, s), 8.42 (1H, s), 8.11 (1H, m), 7.83 (1H, s), 6.66 (1H, s), 4.25 (3H, s), 4.25-4.22 (4H, m), 3.50-3.45 (4H, m).

### EXAMPLE 40

### 2-(4-Acetylpiperazin-1-yl)-7-methoxy-6-oxazol-5-yl-1H-quinolin-4-one

A solution of Example 39 (20 mg, 0.05 mmol) and triethylamine (0.03 ml) in dichloromethane (2 ml) was treated with acetyl chloride (0.01 ml). The mixture was stirred overnight and then washed with saturated aqueous sodium hydrogen carbonate (5 ml) and saturated sodium chloride (5 ml). The organic layer was dried over magnesium sulphate, filtered and the filtrate concentrated in vacuo. The residue was purified by preparative HPLC (method A) to give the title compound (5 mg). MS 368 [M+H]⁺. ¹H-NMR 300 MHz (d₄-MeOH) 8.37 (1H, s), 8.18 (1H, s), 7.43 (1H, s), 7.01 (1H, s), 5.65 (1H, s), 3.95 (3H, s), 3.60-3.75 (4H, m), 3.40-3.55 (4H, m), 2.07 (3H, s).

### EXAMPLE 41

### 3-[4-(7-Methoxy-6-oxazol-5-yl-4-oxo-1,4-dihydroquinolin-2-yl)piperazin-1-yl]propanoic acid methyl ester; formate salt

A mixture of Example 39 (10 mg, 0.025 mmol), triethylamine (0.007 ml) and methyl acrylate (0.5 ml) was stirred at 50° C. for 2 hours. The mixture was concentrated in vacuo and the residue purified by preparative HPLC (method A) to give the title compound (5 mg). MS 413 [M+H]⁺. ¹H-NMR 300 MHz (d₄-MeOH) 8.52 (1H, s), 8.35 (1H, s), 8.25 (1H, s), 7.55 (1H, s), 7.15(1H, s), 5.80 (1H, s), 4.15 (3H, s), 3.75 (3H, s), 3.45-3.60 (4H, m), 2.50-2.85 (8H, m).

### EXAMPLE 42

### 2-[4-(2,2-Dimethylpropyl)piperazin-1-yl]-7-methoxy-6-oxazol-5-yl-1H-quinolin-4-one; formate salt

A mixture of Example 39 (15 mg, 0.037 mmol), triethylamine (0.15 ml), trimethylacetaldehyde (0.06 ml) and 4 Å molecular sieves in THF (10 ml) was stirred at room temperature for one hour. The mixture was then treated with sodium cyanoborohydride (16 mg) and stirred for a further 3 days. The reaction was partitioned between dichloromethane (10 ml) and saturated aqueous sodium hydrogen carbonate (10 ml). The organic layer was dried, filtered and the filtrate concentrated in vacuo. The residue was purified by preparative HPLC (method A) to give the title compound (3 mg). MS 396 [M+H]⁺. ¹H-NMR 300 MHz (d₄-MeOH) 8.45 (1H, s), 8.40 (1H, s), 8.25 (1H, s), 7.55 (1H, s), 7.15(1H, s), 5.75 (1H, s), 4.05 (3H, s), 3.45-3.55 (4H, m), 2.60-2.8 (4H, m), 2.20 (2H, s), 0.95 (9H, s),

The compound of Example 43 was prepared in a similar manner to the compound of Example 42:

### EXAMPLE 43

### 7-Methoxy-6-oxazol-5-yl-2-(4-pyridin-3-ylmethylpiperazin-1-yl)-1H-quinolin-4-one; formate salt

From Example 39 (15 mg, 0.037 mmol), and pyridine-3-carboxaldehyde (0.055 ml). Purification by preparative HPLC (method A) afforded the title compound (3 mg). MS 396 [M+H]⁺. ¹H-NMR 300 MHz (d₄-MeOH) 8.50 (1H, s), 8.45 (1H, s), 8.20 (1H, s), 8.0(1H, s), 7.70-7.8(1H, d), 7.50 (1H, s), 7.25-7.35(1H, d), 6.95 (1H, s), 5.65 (1H, s), 4.00 (3H, s), 3.60 (2H, s), 3.45-3.55 (4H, m), 2.50-2.65 (4H, m).

### EXAMPLE 44

### 2-Azetidin-1-yl-7-methoxy-6-oxazol-5-yl-1H-quinolin-4-one

A mixture of Intermediate 1 (200 mg, 0.5 mmol), azetidine (0.12 g, 1 mmol), THF (10 ml) and mercury (II) chloride (140 mg, 0.5 mmol) were heated to 50° C. for 2 hours. The solvent was removed in vacuo and the residue purified by column chromatography on silica, eluting with 10% methanol/dichloromethane to yield an off-white solid. The solid was treated with diphenyl ether (3 ml) and heated to reflux for 30 minutes. The reaction mixture was diluted with hexane and the precipitate formed collected by filtration. Purification by column chromatography on silica eluting with 10% methanol/dichloromethane afforded the title compound as an off-white solid (20 mg, 13%). TLC R_{f} 0.24 (5% MeOH/DCM). MS 298 [M+H]⁺. ¹H-NMR 300 MHz (d₆-DMSO) 8.20 (1H, s), 8.05 (1H, s), 7.30 (1H, s), 6.85 (1H, s), 5.0 (1H, s), 3.65-3.85 (7H, m), 2.05-2.20 (2H, m).

The compounds of Examples 45-48 were prepared in a similar manner to the compound of Example 44:

### EXAMPLE 45

### 7-Methoxy-6-oxazol-5-yl-2-piperidin-1-yl-1H-quinolin-4-one

From Intermediate 1 (200 mg, 0.5 mmol) and piperidine (0.17 g, 1 mmol). Purification by column chromatography on silica eluting with 10% methanol/dichloromethane afforded the title compound as an off-white solid (79 mg, 49%). TLC R_{f} 0.39 (5% MeOH/DCM). MS 326 [M+H]⁺. ¹H-NMR 300 MHz (d₆-DMSO) 8.30 (1H, s), 8.05 (1H, s), 7.35 (1H, s), 7.00 (1H, s), 5.25 (1H, s), 3.85 (3H, s), 3.10-3.25 (4H, m), 1.35-1.55 (6H, m).

### EXAMPLE 46

### 7-Methoxy-2-((S)-2-methoxymethylpyrrolidin-1-yl)-6-oxazol-5-yl-1H-quinolin-4-one

From Intermediate 1 (200 mg, 0.5 mmol) and (S)-(+)-2-methoxymethylpyrrolidine (65 μl, 1 mmol). Purification by column chromatography on silica eluting with 5-10% methanol/dichloromethane afforded the title compound (1 14 mg). TLC R_{f} 0.49 10%MeOH/DCM. MS 356 [M+H]^{.} ¹H-NMR 300 MHz (d₆-DMSO,130° C.) 8.25 (1H, s), 8.30 (1H, s), 7.40 (1H, s), 7.10(1H, s), 4.25(1H, s, br), 4.05 (3H, s), 3.60-3.45(4H, m), 3.35 (3H, s), 2.10-1.95 (4H, m).

### EXAMPLE 47

### 3-(7-Methoxy-6-oxazol-5-yl-4-oxo-1,4-dihydroquinolin-2-ylamino) benzonitrile

From Intermediate 1 (200 mg, 0.5 mmol) and 3-aminobenzonitrile (0.12 g, 1 mmol). Purification by column chromatography on silica eluting with 0-5% methanol/dichloromethane afforded the title compound as a pale orange solid (6.4 mg, 19%). TLC R_{f} 0.25 (5% MeOH/DCM). MS 359 [M+H]⁺. ¹H-NMR 300 MHz (d₆-DMSO) 11.3 (1H, s, br), 9.32 (1H, s, br), 8.31 (1H, s), 8.12 (1H, s), 7.98-7.91 (1H, m), 7.41 (1H, s), 7.38-7.31 (1H, m), 7.31-7.27 (1H, m), 7.02 (1H, s), 6.17-6.12 (1H, m), 3.92 (3H, s), 3.60 (4H, m), 3.30-3.40 (4H, m), 3.78 (3H, s), 1.35-1.25 (6H, s).

### EXAMPLE 48

### 7′-Methoxy-6′-oxazol-5-yl-3,4-dihydro-2H,1′H-[1,2]biquinolinyl-4′-one

From Intermediate 1 (200 mg, 0.5 mmol), 1,2,3,4-tetrahydroquinoline (0.1 ml, 0.8 mmol) and mercury (II) chloride. The mixture was stirred at room temperature overnight. Purification by column chromatography on silica eluting with 1% methanol/dichloromethane gave the desired intermediate along with an unidentified impurity. The mixture was dissolved in diphenyl ether and heated at 250° C. for approximately 30 minutes. The reaction mixture was diluted with heptane and filtered. The resulting solid was purified by column chromatography on silica eluting with 5% methanol/dichloromethane to afford the title compound as a brown solid (120 mg, 63%). HPLC RT 2.32 mins. MS 374 [M+H]⁺. ¹H-NMR 300 MHz (d₄-MeOH) 8.55 (1H, s), 8.25 (1H, s), 7.55 (1H, s), 7.23-7.00 (5H, m), 6.00 (1H, s), 4.08 (3H, s), 3.85-3.78 (2H, m), 2.90-2.85 (2H, m), 2.12-2.02 (2H, m).

### EXAMPLE 49

### 7-Methoxy-2-(1-methyl-1H-pyrazol-3-ylamino)-6-oxazol-5-yl-1H-quinolin-4-one

A mixture of Intermediate 1 (300 mg, 0.8 mmol) and 3-amino-1-methyl pyrazole (0.1 g, 1 mmol) in DMF (4 ml) was heated to 60° C. for 1 hour and then at 100° C. for a further 2 hours. Water (20 ml) was added and the mixture was allowed to stand overnight. The aqueous mixture was extracted with dichloromethane (3×10 ml) and the dichloromethane fractions washed with saturated sodium hydrogen carbonate solution (10 ml). The organic layer was dried over magnesium sulphate, filtered and the solvent was removed in vacuo to give a brown oil. The oil was dissolved in diphenyl ether (4 ml) and heated to 195° C. for 10 minutes. The reaction mixture was diluted with hexane to give an oil. The diphenyl ether/hexane solution was decanted off and the oil dissolved in dichloromethane. The solvent was removed in vacuo and the residue purified by preparative HPLC (method A) to yield the title compound as a pale yellow solid (0.5 mg, 0.2%). TLC R_{f} 0.25 (5% MeOH/DCM). MS 359 [M+H]⁺.

### EXAMPLE 50

### 7-Methoxy-6-oxazol-5-yl-2-pyrrolidin-1-yl-1H-quinolin-4-one

A mixture of Intermediate 1 (200 mg, 0.5 mmol), pyrrolidine (70 mg, 1 mmol) in diphenyl ether (4 ml) was heated to 50° C. for 2 hours and then at 195° C. for 30 minutes. The reaction mixture was diluted with hexane to give an off-white solid, which was filtered off. Purification by column chromatography on silica eluting with 10% methanol/dichloromethane afforded the title compound (76 mg, 48%). TLC R_{f} 0.37 (5% MeOH/DCM). MS 312 [M+H]⁺. ¹H-NMR 300 MHz (d₆-DMSO) 8.42 (1H, s), 8.20 (1H, s), 7.45 (1H, s), 7.25 (1H, s), 5.10 (1H, s), 3.95 (3H, s), 3.25-3.45 (4H, m), 1.85-2.00 (4H, m).

### EXAMPLE 51

### 2-Benzylamino-7-methoxy-6-oxazol-5-yl-1H-quinolin-4-one

To a mixture of Intermediate 1 (200 mg, 0.5 mmol), benzylamine (0.10 g, 1 mmol) in diphenyl ether (4 ml) was added mercury (II) chloride (140 mg, 0.5 mmol). The mixture was heated to 50° C. for 2 hours and then to reflux for 30 minutes. The reaction mixture was diluted with hexane and the precipitate formed collected by filtration. Purification by column chromatography on silica eluting with 5% methanol/dichloromethane, followed by preparative HPLC (method A) afforded the title compound as an off-white solid (20 mg, 12%). TLC R_{f} 0.42 (5% MeOH/DCM). MS 348 [M+H]⁺. ¹H-NMR 300 MHz (d₄-MeOH) 8.52 (1H, s), 8.15 (1H, s), 7.5 (1H, s), 7.20-7.40 (5H, m), 6.90 (1H, s), 5.60 (1H, s), 4.45 (2H, s), 4.05 (3H, s).

### EXAMPLE 52

### 1-(7-Methoxy-6-oxazol-5-yl-4-oxo-1,4-dihydroquinolin-2-yl)piperidine-4-carboxylic acid

A solution of Example 9 (150 mg) in THF (10 ml), methanol (2 ml) and water (2 ml) was treated with lithium hydroxide monohydrate (10 mg) and stirred at room temperature 16 hours. The organic solvents were removed in vacuo and the aqueous residue was acidified with acetic acid. All solvents were removed in vacuo and the residue purified by column chromatography on silica eluting with 10-20% methanol/dichloromethane to give the title compound as an off white solid (46 mg, 32%). TLC R_{f} 0.22 (20% MeOH/DCM). HPLC RT 1.72 mins. MS 370 [M+H]⁺.

### EXAMPLE 53

### 1-(7-Methoxy-6-oxazol-5-yl-4-oxo-1,4-dihydroquinolin-2-ylamino)butyric acid

To a suspension of Example 12 (25 mg) in methanol (5 ml) and water (5 ml) was added sodium hydroxide (15 mg). The reaction mixture was then heated to reflux for 5 hours. The solution was acidified using 2N hydrochloric acid and the resulting mixture concentrated in vacuo. The resulting solid suspension was filtered off, washed with water and diethyl ether and dried in a vacuum oven to give the title compound as an off-white solid (10 mg). HPLC RT 1.63 mins. MS 344 [M+H]⁺.

The compound of Example 54 was prepared in a similar manner to Example 53:

### EXAMPLE 54

### 3-[(7-Methoxy-6-oxazol-5-yl-4-oxo-1,4-dihydroquinolin-2-ylamino)-methyl]benzoic acid

From Example 21 (246 mg) to give the title compound as a tan solid (224 mg, 94%). HPLC RT 1.87 mins. MS 392 [M+H]⁺.

### EXAMPLE 55

### N-Furan-2-ylmethyl-4-(7-methoxy-6-oxazol-5-yl-4-oxo-1,4-dihydro-quinolin-2-ylamino)butyramide

Example 53 (50 mg, 0.146 mmol), DMF (10 ml), 1-(3-dimethylaminopropyl)-3-ethyl carbodiimde hydrochloride (34 mg, 0.175 mmol), 1-hydroxybenzotriazole hydrate (24 mg, 0.175 mmol) and furfurylamine (0.015 ml, 0.175 mmol) were combined under a nitrogen atmosphere at room temperature. The resulting solution was stirred at room temperature for 5 hours. The solvents were removed in vacuo and the resulting residue purified by preparative HPLC (method A) to give the title compound as a cream solid (27 mg, 44%). HPLC RT 1.97 mins. MS 423 [M+H]⁺.

Examples 56-63 were prepared in a similar manner to Example 55:

### EXAMPLE 56

### 7-Methoxy-2-(4-morpholin-4-yl-4-oxo-butylamino)-6-oxazol-5-yl-1H-quinolin-4-one; formate salt

From Example 53 (50 mg, 0.146 mmol) and morpholine (0.02 ml, 0.219 mmol). Purification by preparative HPLC (method A) to give the title compound as an off-white solid (28 mg). HPLC RT 1.83 mins. MS 413 [M+H]⁺.

### EXAMPLE 57

### 4-(7-Methoxy-6-oxazol-5-yl-4-oxo-1 4-dihydro-quinolin-2-ylamino)-N-(2-morpholin-4-yl-ethyl)butyramide; formate salt

From Example 53 (50 mg, 0.146 mmol) and 4-(2-aminoethyl)morpholine (0.03 ml, 0.219 mmol). Purification by preparative HPLC (method B) afforded the title compound as an off-white solid (14 mg). HPLC RT 1.32 mins. MS 456 [M+H]⁺.

### EXAMPLE 58

### 4-(7-Methoxy-6-oxazol-5-yl-4-oxo-1 4-dihydro-quinolin-2-ylamino)-N-methyl-butyramide; formate salt

From Example 53 (50 mg, 0.146 mmol), methylamine hydrochloride (50 mg, 0.729 mmol) and triethylamine (0.1 ml, 0.729 mmol). Purification by preparative HPLC (method A) followed by trituration with hot dichloromethane/ethyl acetate twice afforded the title compound as an off white solid (4 mg). HPLC RT 1.69 mins. MS 357 [M+H]⁺.

### EXAMPLE 59

### 7-Methoxy-2-[3-(morpholine-4-carbonyl)benzylamino]-6-oxazol-5-yl-1H-quinolin-4-one

From Example 54 (21 mg, 0.054 mmol) and morpholine (0.01 ml, 0.081 mmol). Purification by preparative HPLC (method B then method C) gave the title compound as an off-white solid (3 mg). HPLC RT 1.86 mins. MS 461 [M+H]⁺.

### EXAMPLE 60

### 3-[(7-Methoxy-6-oxazol-5-yl-4-oxo-1,4-dihydro-quinolin-2-ylamino)-methyl]-N-methylbenzamide

From Example 54 (21 mg, 0.054 mmol), methylamine hydrochloride (10 mg) and triethylamine (0.1 ml). Purification by preparative HPLC (method B then method C) afforded the title compound as an off-white solid (0.5 mg). HPLC RT 1.80 mins. MS 405 [M+H]⁺.

### EXAMPLE 61

### 4-[3-[(7-Methoxy-6-oxazol-5-yl-4-oxo-1,4-dihydro-quinolin-2-ylamino)-methyl]benzoylamino]piperidine-1-carboxylic acid tert-butyl ester

From Example 54 (32 mg), Boc-(4-amino)-piperidine hydrochloride (20 mg) and triethylamine (0.1 ml). Purification by preparative HPLC (method B then method C) yielded the title compound as an off-white solid (1 mg). HPLC RT 2.40 mins. MS 574 [M+H]⁺.

### EXAMPLE 62

### 3-[(7-Methoxy-6-oxazol-5-yl-4-oxo-1,4-dihydro-quinolin-2-ylamino)-methyl]-N-pyridin-2-ylmethylbenzamide

From Example 54 (44 mg) and 2-(aminomethyl)pyridine (0.02 ml). Purification by preparative HPLC (method C) afforded the title compound as an off white solid (26 mg). HPLC RT 1.69 mins. MS 482 [M+H]⁺.

### EXAMPLE 63

### 3-[(7-Methoxy-6-oxazol-5-yl-4-oxo-1,4-dihydro-quinolin-2-ylamino)-methyl]-N-(2-morpholin-4-yl-ethyl)benzamide; acetate salt

From Example 54 (37 mg) and 4-(2-aminoethyl)morpholine (0.03 ml). Purification by preparative HPLC (method C) afforded the title compound as an off-white solid (28 mg). HPLC RT 1.46 mins. MS 504 [M+H]⁺.

### EXAMPLE 64

### N-(7-Methoxy-6-oxazol-5-yl-4-oxo-1,4-dihydro-quinolin-2-yl)methane sulfonamide

To a stirred solution of Intermediate 41 (0.02 g, 0.08 mmol) in dichloromethane (5 ml), was added methane sulfonyl chloride (9 mg, 0.008 mmol) followed by pyridine (0.1 ml). After 2 hours the reaction mixture was taken up in dilute acetic acid (5 ml) and extracted into dichloromethane (20 ml). The organic layer was washed with saturated aqueous sodium chloride (10 ml), separated, dried over magnesium sulphate, flitered and the solvent removed in vacuo. The residue was washed with dichloromethane and ethyl acetate, then dried in a vacuum oven overnight, to give the title compound (5 mg, 19%). TLC R_{f} 0.54 (10% MeOH/DCM). MS 336 [M+H]⁺. ¹H-NMR 300 MHz (d₄MeOH) 8.18 (1H, s), 8.12 (1H, s), 7.54 (1H, s), 7.04 (1H, s), 6.75 (1H, s), 3.96 (3H, s), 3.38 (3H, s).

The compounds of Examples 65-66 were prepared in a similar manner to the compound of Example 64:

### EXAMPLE 65

### 1-Ethyl-3-(7-methoxy-6-oxazol-5-yl-4-oxo-1,4-dihydroquinolin-2-yl)urea

Intermediate 41 (20 mg), ethyl isocyanate (0.2 ml) DMF (5 ml) and dichloromethane (5 ml) were combined under a nitrogen atmosphere and heated to 84° C. for 3hours. The solvents were removed in vacuo and the residue triturated with dichloromethane. Purification by preparative HPLC (method A) afforded the title compound as an off-white solid (4 mg). HPLC RT 2.03 mins. MS 329 [M+H]⁺.

### EXAMPLE 66

### N-(7-Methoxy-6-oxazol-5-yl-4-oxo-1,4-dihydroquinolin-2-yl)acetamide

Intermediate 41 (80 mg), acetic anhydride (32 mg) and DMF (5 ml) were combined under a nitrogen atmosphere and heated to 90° C. for 16 hours. The reaction mixture was evaporated in vacuo and the residue purified by preparative HPLC (method A) to give the title compound as an off-white solid (3 mg). HPLC RT 1.86 mins. MS 300 [M+H]⁺.

### EXAMPLE 67

### 2-(Benzylmethylamino)-7-methoxy-6-oxazol-5-yl-1H-quinoline-4-thione

A suspension/solution of Example 34 (100 mg, 0.28 mmol) in dry toluene (10 ml) was treated with Lawesson's reagent (134 mg, 0.33 mmol) and the mixture heated at reflux overnight. The solvent was removed in vacuo and the residue purified by column chromatography on silica eluting with 0-20% methanol/dichloromethane followed by preparative HPLC (method A) to give the title compound as a yellow solid (1 mg, 1%). HPLC RT 2.85 mins. MS 378 [M+H]⁺.

The ability of the compounds of the invention to inhibit the IMPDH enzymes may be determined using the following assays: Abbreviatons used:

| | |
|---|---|
| IMPDH | Inosine 5′monophosphate dehydrogenase |
| IMP | Inosine 5′monophosphate |
| XMP | Xanthosine 5′-monophosphate |
| NAD | β- Nicotinamide adenine dinucleotide |
| NADH | β- Nicotinamide adenine dinucleotide, reduced form |
| MTT | 3-(4,5-Dimethylthiazol-2-yl)-2,5-diphenyltetrazolium |
| | bromide |

Assay Protocol 1

IMPDH catalyses the NAD dependent oxidation of IMP to XMP with concomitant reduction of the coenzyme. IMPDH activity was determined by monitoring the production of the fluorescent product, NADH. Assays were performed in a final volume of 200 μl containing IMPDH (2 μg), NAD (100 μM), IMP (100 μM), 1% DMSO, 30 mM KCl and 100 mM Tris/HCl, pH7.5. Fluorescence (excitation 340 nm/emission 465 nm) was read continuously at 25° C. for 30 minutes. From this data, initial rates (i.e. change in fluorescence intensity per minute) were calculated. To determine the IC₅₀ values, test compounds were prepared at an initial concentration of 1.0 mM in 100% DMSO, then diluted in assay buffer to 0.2 mM. Further dilutions were made in assay buffer containing 20% DMSO, prior to diluting 20-fold into the assay, to allow testing across the range 0.3 nM to 10 μM.

The functional effect of the compounds of the invention may be demonstrated using the following assay:

PBMC Proliferation Assay

Peripheral blood mononuclear cells were isolated from freshly taken human blood using standard procedures. Cells were plated out in RPMI medium containing 5% human serum in the presence and absence of inhibitor. PHA (25 μl of 30 μg/ml solution to each well) was added and the plates were incubated at 37° C. in an atmosphere of 95% air/5% CO₂ for 48 hours. 0.5 μCi of tritiated thymidine was added to each well and the plates were incubated for a further 18 hours. The contents of the plate were transferred to a filter plate and the cells washed with saline. The plates were dried, microscintillation fluid was added to each well and the plate was counted on a scintillation counter. IC₅₀ values were calculated by plotting inhibitor concentration versus %inhibition. The assay described above can be carried out using anti-CD3 (40 μl of 3750 ng/ml concentration to each well) stimulation instead of PHA.

Compounds of the invention such as compounds of the Examples inhibit IMPDH enzymes with IC₅₀ values of 5 μM or below.

1. A compound of formula (1): 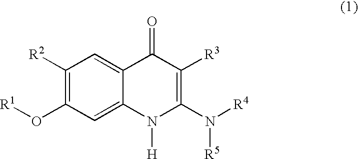
wherein:
X is an O or S atom;
R¹ is an aliphatic, cycloaliphatic or cycloalkyl-alkyl-group;
R² is a —CN group or an optionally substituted heteroaromatic group;
R³ is a hydrogen atom or an alkyl, —CN, —CO₂H, —CO₂R⁶ or —CONR⁷R⁸ group, in which R⁶ is an alkyl group and R⁷ and R⁸, which may be the same or different, is each a hydrogen atom or an alkyl group;
R⁴ is a chain -Alk¹-L¹-Alk²-R⁹ in which Alk¹ is a covalent bond or an optionally substituted aliphatic chain, L¹ is a covalent bond or a linker atom or group, Alk² is a covalent bond or a C₁₋₃ alkylene chain and R⁹ is a hydrogen atom or an optionally substituted cycloaliphatic, heterocycloaliphatic, aromatic or heteroaromatic group; provided that R⁴ is not a hydrogen atom;
R⁵ is a hydrogen atom or an alkyl group;
or NR⁴R⁵ forms an optionally substituted heterocycloaliphatic ring optionally fused to an optionally substituted monocyclic C₆₋₁₂aromatic group or an optionally substituted monocyclic C₁₋₉heteroaromatic group;
and the salts, solvates, hydrates, tautomers, isomers or N-oxides thereof;
provided that the compound of formula (1) is other than:
7-methoxy-2-methylamino-6-oxazol-5-yl-1H-quinolin-4-one or
2-dimethylamino-7-methoxy-6-oxazol-5-yl-1H-quinolin-4-one.

2. A compound according to claim 1 which has the formula (2): 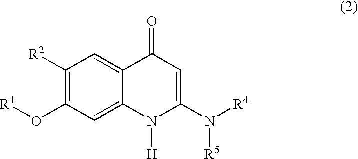
wherein R¹, R², R⁴ and R⁵ are as defined in claim 1.

3. A compound according to claim 1, wherein R¹ is a C₁₋₆ alkyl group.

4. A compound according to claim 3, wherein R¹ is a methyl group.

5. A compound according to claim 1, wherein R² is an optionally substituted heteroaromatic group.

6. A compound according to claim 5, wherein R² is a five-membered heteroaromatic group containing one, two, three or four heteroatoms selected from oxygen, sulfur or nitrogen.

7. A compound according to claim 6, wherein R² is an oxazole group.

8. A compound according to claim 1, wherein R⁴ is the chain -Alk¹-R⁹.

9. A compound according to claim 1, wherein Alk¹, L¹ and Alk² is each a covalent bond and R⁹ is an optionally substituted phenyl or monocyclic heteroaromatic group.

10. A compound according to claim 9, wherein R⁹ is an optionally substituted phenyl, pyridyl, pyrimidinyl, pyridazinyl or pyrazinyl group.

11. A compound according to claim 1 wherein Alk¹ is an optionally substituted aliphatic chain, L¹ and Alk² is each a covalent bond and R⁹ is an optionally substituted cycloaliphatic, heterocycloaliphatic, aromatic or heteroaromatic group.

12. A compound according to claim 11 wherein Alk¹ is a C₁₋₃ alkylene chain.

13. A compound according to claim 11 wherein R⁹ is optionally substituted azetidinyl, pyrrolidinyl, pyrrolidinonyl, piperidinyl, imidazolidinyl, thiazolidinyl, piperazinyl, N—C₁₋₆ alkylpiperazinyl, especially N-methyl piperazinyl, N—C₁₋₆alkylpyrrolidinyl, especially N-methylpyrrolidinyl, N—C₁₋₆ alkylpiperidinyl, especially N-methylpiperidinyl, homopiperazinyl, morpholinyl, thiomorpholinyl, oxazolidinyl, tetrahydrofuranyl, tetrahydropyranyl, phenyl, pyrrolyl, furyl, thienyl, imidazolyl, N—C₁₋₆ alkylimidazolyl, oxazolyl, isoxazolyl, thiazolyl, isothiazolyl, pyrazolyl, triazolyl, oxadiazolyl, thiadiazolyl, pyridyl, pyrimidinyl, pyridazinyl, pyrazinyl, tetrazolyl or triazinyl.

14. A compound according to claim 11 wherein R⁹ is an optionally substituted C₃₋₆ cycloalkyl group.

15. A compound according to claim 1 wherein R⁵ is a hydrogen atom or a methyl group.

16. A compound according to claim 1 wherein NR⁴R⁵ forms an optionally substituted heterocycloaliphatic group.

17. A compound according to claim 16 wherein NR⁴R⁵ is an optionally substituted azetidinyl, pyrrolidinyl, piperidinyl, piperazinyl, N—C₁₋₆ alkylpiperazinyl, homopiperazinyl, morpholinyl or thiomorpholinyl group.

18. A compound according to claim 17 wherein NR⁴R⁵ is an optionally substituted pyrrolidinyl or piperidinyl group.

19. A compound according to claim 16 wherein NR⁴R⁵ is fused to an optionally substituted phenyl or five or six membered heteroaryl group.

20. A compound according to claim 19 in which NR⁴R⁵ is an optionally substituted 2,3-dihydro-1H-indolyl, 2,3-dihydro-1H-isoindolyl, 1,2,3,4 tetrahydroquinolinyl or 1,2,3,4 tetrahydroisoquinolinyl group.

21. A compound which is:
2-(2,3-Dihydroindol-1-yl)-7-methoxy-6-oxazol-5-yl-1H-quinolin-4-one;
2-(Dihydro-1H-isoquinolin-2-yl)-7-methoxy-6-oxazol-5-yl-1H-quinolin-4-one;
2-(1,3-Dihydroisoindol-2-yl)-7-methoxy-6-oxazol-5-yl-1H-quinolin-4-one;
2-(6,7-Dimethoxy-3,4-dihydro-1H-isoquinolin-2-yl)-7-methoxy-6-oxazol-5-yl-1H-quinolin-4-one;
2-(5-Bromo-2,3-dihydroindol-1-yl)-7-methoxy-6-oxazol-5-yl-1H-quinolin-4-one;
7-Methoxy-2-(2-methyl-2,3-dihydroindol-1-yl)-6-oxazol-5-yl-1H-quinolin-4-one;
7′-Methoxy-6′-oxazol-5-yl-3,4-dihydro-2H, 1′H-[1,2′]biquinolinyl-4′-one;
and the salts, solvates, hydrates, tautomers, isomers or N-oxides thereof.

22. A pharmaceutical composition comprising a compound according to claim 1, together with one or more pharmaceutically acceptable carriers, excipients or diluents.

23. Use of a compound of claim 1, for the treatment of cancer, inflammatory disorders, autoimmune disorders, psoriatic disorders and viral disorders.
